(19) United States
(12) Patent Application Publication
(10) Pub. No.: US 2026/0273259 A1
(43) Pub. Date: Sep. 17, 2026

(54) SYSTEMS FOR FACILITATING THE MANAGEMENT AND CREATION OF TATTOOS

(71) Applicants: JOSHUA FRANCISCO ARCEO, LAS VEGAS, NV (US); Cash Viedt, LAS VEGAS, NV (US); FRANCISCO DEL ROSARIO ARCEO, LAS VEGAS, NV (US)

(72) Inventors: JOSHUA FRANCISCO ARCEO, LAS VEGAS, NV (US); Cash Viedt, LAS VEGAS, NV (US); FRANCISCO DEL ROSARIO ARCEO, LAS VEGAS, NV (US)

(21) Appl. No.: 19/678,963

(22) Filed: May 15, 2026

(30) Foreign Application Priority Data
Aug. 25, 2022 (CN) 202211028570.2

(51) Int. Cl. A61M 37/00

(52) U.S. Cl. CPC A61M 37/0076; A61M 2205/106; A61M 2205/3306; A61M 2205/50

(57) ABSTRACT

The present disclosure provides a system for facilitating the management and creation of tattoos. Further, the system may include a processor configured for obtaining one or more information associated with the creating of one or more tattoos, analyzing the one or more information, determining one or more operations for the creating of the one or more tattoos based on the analyzing of the one or more information, and generating one or more operation data based on the determining of the one or more operations. Further, the system may include a communication interface configured for transmitting the one or more operation data to one or more devices. Further, the one or more devices may be configured for performing one or more device operations corresponding to the one or more operations for facilitating the creating of the one or more tattoos based on the one or more operation data.

### FIELD OF DISCLOSURE

The present disclosure relates to tattooing systems, adaptive control systems, augmented reality guidance systems, and real-time sensor-based control of tattooing devices. More specifically, the present disclosure relates to systems for facilitating the management and creation of tattoos.

### BACKGROUND

Tattooing technologies occupy an important position at the intersection of body art, precision handheld instrumentation, human factors engineering, and skin-interaction control. Progress in this field carries significance for artistic fidelity, procedure safety, repeatability of outcomes, and efficient execution across a wide range of anatomical locations and skin conditions. Advancements in tattoo-related devices and operating workflows can affect not only the quality and consistency of tattoo application, but also practitioner confidence, client experience, and the ability to execute increasingly complex designs with accuracy. As tattoo procedures involve repeated mechanical interaction with living tissue, continued development in this technical area remains relevant to both performance and risk management.

In the given context, a desirable aspect would be to facilitate more precise and reliable tattoo execution while maintaining consistent alignment between intended artwork and actual application on a target skin surface. It would further be desirable to improve control over procedure variables that influence line quality, shading uniformity, pigment placement, tissue response, and overall visual accuracy. Achieving such an objective would represent meaningful progress because tattoo application often depends on maintaining fidelity to a desired design under conditions involving motion, curvature, localized deformation, and changing interaction characteristics at the skin interface.

Existing approaches directed to this objective present multiple technical difficulties. One difficulty arises from the dependence on manual selection and adjustment of operating parameters during tattooing. Variations in skin thickness, elasticity, hydration, contour, and localized resistance can alter needle-tissue interaction in ways that are difficult to evaluate and compensate for continuously during a procedure. As a result, maintaining uniform penetration, pigment distribution, and visual consistency across different regions of a subject's body can be challenging.

Another difficulty relates to design placement and alignment. When artwork is positioned on a skin surface before execution, maintaining accurate correspondence between the intended design and the actual target location can be hindered by body curvature, posture changes, breathing-related motion, muscular movement, skin stretching, and compression during handling or procedure setup. Distortion, fading, smearing, partial loss of reference markings, or positional drift can reduce placement accuracy and make it more difficult to preserve intended proportions and spatial relationships throughout the tattooing process.

Further difficulties arise from limited procedural feedback during active use. A practitioner may have incomplete visibility into changing interaction conditions between the tattoo instrument and the skin, including shifts in resistance, vibration behavior, ink uptake, saturation behavior, or other execution-related conditions that can influence outcome quality or tissue stress. In the absence of timely and usable feedback, deviations from an intended path, excessive reworking of an area, inconsistent fill density, or undesirable tissue effects may be identified only after they have already affected the tattoo.

Additional challenges involve maintaining procedural consistency across different tattooing techniques and operator behaviors. Differences in hand speed, dwell time, directional changes, and movement style can influence the visual result and may require responsive compensation to preserve desired output. Where equipment and visual references operate as separate, non-coordinated tools, workflow fragmentation can increase cognitive burden on the practitioner and make it more difficult to sustain accuracy, efficiency, and repeatability during extended or technically demanding sessions.

Therefore, there is a need for improved systems for facilitating the management and creation of tattoos that may overcome one or more of the preceding problems.

SUMMARY OF DISCLOSURE

This summary is provided to introduce a selection of concepts in a simplified form that is further described below in the Detailed Description. This summary is not intended to identify key features or essential features of the claimed subject matter. Nor is this summary intended to be used to limit the claimed subject matter's scope.

The present disclosure provides a system for facilitating the management and creation of tattoos. Further, the system may include a processor. Further, the processor may be configured for obtaining one or more information associated with the creating of one or more tattoos on a portion of a skin of a body part of a client. Further, the processor may be configured for analyzing the one or more information. Further, the processor may be configured for determining one or more operations for the creating of the one or more tattoos based on the analyzing of the one or more information. Further, the processor may be configured for generating one or more operation data associated with the one or more operations based on the determining of the one or more operations. Further, the system may include a communication interface communicatively coupled with the processor. Further, the communication interface may be configured for transmitting the one or more operation data to one or more devices. Further, the one or more devices may be configured for performing one or more device operations corresponding to the one or more operations for facilitating the creating of the one or more tattoos based on the one or more operation data.

The present disclosure provides a system for facilitating the management and creation of tattoos. Further, the system may include a processor. Further, the processor may be configured for obtaining one or more information associated with the creating of one or more tattoos on a portion of a skin of a body part of a client. Further, the one or more information includes one or more first sensor data representing one or more attributes of one or more tattooing devices relative to the skin of the body part of the client. Further, the processor may be configured for analyzing the one or more information. Further, the analyzing of the one or more information includes analyzing the one or more first sensor data. Further, the processor may be configured for determining one or more operations for the creating of the one or more tattoos based on the analyzing of the one or more information. Further, the one or more operations include one or more tattooing device operations associated with the one or more tattooing devices. Further, the processor may be configured for identifying one or more skin interaction parameters based on the analyzing of the one or more first sensor data. Further, the processor may be configured for generating one or more control data for the one or more tattooing devices based on the identifying of the one or more skin interaction parameters. Further, the processor may be configured for generating one or more operation data associated with the one or more operations based on the determining of the one or more operations. Further, the one or more operation data include the one or more control data. Further, the system may include a communication interface communicatively coupled with the processor. Further, the communication interface may be configured for transmitting the one or more operation data to one or more devices. Further, the one or more devices include the one or more tattooing devices. Further, the one or more tattooing devices include one or more first sensors which may be configured for generating the one or more first sensor data. Further, the one or more devices may be configured for performing one or more device operations corresponding to the one or more operations for facilitating the creating of the one or more tattoos based on the one or more operation data. Further, the one or more device operations include the one or more tattooing device operations.

Further, the performing of the one or more device operations includes performing the one or more tattooing device operations based on the one or more control data.

The present disclosure provides a tattooing system for facilitating the design, alignment, and execution of tattoos. The tattooing system includes a handheld tattoo device having a motor-driven reciprocating needle assembly and a stroke-adjustment mechanism operatively coupled to the needle assembly. One or more sensors detect interaction conditions between the tattoo device and a target skin surface, including resistance, displacement, vibration, and motion. A control mechanism receives sensor measurements, identifies changes in skin interaction conditions, and generates control signals that actuate the stroke-adjustment mechanism and modify operating parameters including stroke length, frequency, voltage, torque, and penetration depth. The control mechanism performs continuous parameter adjustment during active needle insertion into the skin surface in a closed-loop configuration and maintains consistent needle penetration across varying skin resistance. The control mechanism operates with a response latency suitable for real-time control between receipt of sensor data and generation of control signals.

The tattooing system includes one or more sensors that generate sensor data representing operational characteristics and interaction conditions relative to a skin surface. A control mechanism receives the sensor data, identifies resistance changes and deformation characteristics, and generates control signals that continuously adjust operation of the tattoo device during active tattooing. The control mechanism actuates one or more mechanisms that modify stroke length, frequency, voltage, torque, and penetration depth in real time, wherein operations occur continuously in a closed-loop configuration during needle insertion into the skin surface.

The tattooing system includes a wearable augmented reality device comprising one or more sensors that generate visual and spatial data representing a target skin surface. A stencil generation mechanism produces a digital stencil spatially registered to the skin surface. A synchronization mechanism maintains alignment of the digital stencil relative to the skin surface and continuously updates stencil position based on movement and deformation. The tattooing system synchronizes stencil alignment with operation of the tattoo device in real time, wherein stencil-derived positional data influences operation of the tattoo device and sensor data from the tattoo device influences stencil alignment.

Both the foregoing summary and the following detailed description provide examples and are explanatory only. Accordingly, the foregoing summary and the following detailed description should not be considered to be restrictive. Further, features or variations may be provided in addition to those set forth herein. For example, embodiments may be directed to various feature combinations and sub-combinations described in the detailed description.

### BRIEF DESCRIPTIONS OF DRAWINGS

The accompanying drawings, which are incorporated in and constitute a part of this disclosure, illustrate various embodiments of the present disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the applicants. The applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure.

The drawings presented with this disclosure may illustrate representative and non-limiting arrangements of hardware components, software modules, artificial intelligence subsystems, machine learning architectures, data processing pipelines, user interfaces, network topologies, and memory arrangements that may be used to understand embodiments of the present subject matter. They may depict functional or conceptual layouts intended to facilitate the explanation of the disclosed principles. The geometric appearance, dimensional proportions, ordering, grouping, and naming of elements within the drawings are not intended to imply any restriction on implementation. The drawings may schematically portray computing environments containing client devices, servers, distributed computing clusters, communication networks, storage systems, or artificial intelligence models arranged for training, inference, or combined operations. The drawings may include simplified symbolic representations of algorithmic processes, workflows, blocks, or modules; such symbolic representations are treated as abstractions of underlying hardware and software operations rather than literal structural requirements. Similarly, lines connecting components may represent logical associations, communication pathways, or data relationships rather than any specific physical wiring or layout. These figures may also illustrate non-exhaustive examples of operational stages, sequencing, or interactions among artificial intelligence components such as encoders, decoders, generators, discriminators, featurizers, transformers, or safety-validation modules. Any specific combination or configuration shown is presented for explanatory clarity only. Additional drawings, alternative views, or more granular depictions may be used without affecting the scope of the claims.

FIG. 1 is an illustration of an online platform 100 consistent with various embodiments of the present disclosure.

FIG. 2 is a block diagram of a computing device 200 for implementing the methods disclosed herein, in accordance with some embodiments.

FIG. 3 is a block diagram of a machine-learning system 300 for implementing various embodiments of this disclosure, in accordance with some embodiments.

FIG. 4 illustrates a block diagram of a system 400 facilitating the management and creation of tattoos, in accordance with some embodiments.

FIG. 5 illustrates a block diagram of the system 400 facilitating the management and creation of tattoos, in accordance with some embodiments.

FIG. 6 illustrates a block diagram of the system 400 facilitating the management and creation of tattoos, in accordance with some embodiments.

FIG. 7 illustrates a block diagram of the system 400 facilitating the management and creation of tattoos, in accordance with some embodiments.

FIG. 8 illustrates a block diagram of the system 400 facilitating the management and creation of tattoos, in accordance with some embodiments.

FIG. 9 illustrates a block diagram of the system 400 facilitating the management and creation of tattoos, in accordance with some embodiments.

FIG. 10 illustrates a block diagram of the system facilitating the management and creation of tattoos, in accordance with some embodiments.

FIG. 11 illustrates a block diagram of a system 1100 facilitating the management and creation of tattoos in accordance with some embodiments.

FIG. 12 illustrates block diagram of a tattooing system 1200, in accordance with some embodiments.

FIG. 13A illustrates a tattooing device 1302 associated with the system 400, in accordance with some embodiments.

FIG. 13B illustrates a visualization device 1304 associated with the system 400, in accordance with some embodiments.

### DETAILED DESCRIPTION OF DISCLOSURE

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing herefrom, which scope is to be defined by the claims and the equivalents thereof. It is not intended that the scope of patent protection be defined by reading into any claim limitation found herein and/or issuing herefrom that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

The embodiments described herein are non-limiting and may include additional, fewer, or alternative components, arrangements, or operational sequences without departing from the scope of the present disclosure.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term-differs in any way from any particular dictionary definition of such term, it is intended that the meaning of the term as understood by the ordinary artisan should prevail.

Furthermore, it is important to note that, as used herein, “a” and “an” each generally denote “at least one,” and/or “one or more,” but do not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings.

Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the claims found herein and/or issuing herefrom. The present disclosure contains headers. It should be understood that these headers are used as references and are not to be construed as limiting upon the subject matter disclosed under the header.

The present disclosure includes many aspects and features. Moreover, while many aspects and features relate to, and are described in the context of the disclosed use cases, embodiments of the present disclosure are not limited to use only in this context.

In some embodiments, the present disclosure includes computing, artificial intelligence, and data processing frameworks used in connection with the tattooing system. These frameworks may include machine learning models, data processing pipelines, and distributed or localized computing architectures used to process sensor data, visual data, and operational data. The foregoing computing and artificial intelligence frameworks are illustrative and are not required for operation of the tattooing system described herein, which may operate using embedded control logic and localized processing independent of distributed computing architectures.

The present disclosure contemplates implementations involving artificial intelligence, machine learning, distributed computation, and computer-implemented systems operating upon data represented as physical electronic or optical signals. Descriptions of processing, analyzing, determining, transforming, encoding, decoding, generating, inferring, synthesizing, modifying, storing, retrieving, ranking, filtering, validating, classifying, or otherwise manipulating information are to be understood as referring to the actions of computing systems, computing devices, electronic devices, or computational circuits that manipulate such signals in memory elements, registers, buffers, or storage media.

The disclosure contemplates implementations in which artificial intelligence systems perform perception, synthesis, inference, prediction, or generation of information and/or data using models whose configurations may evolve based on training, feedback, or adaptive learning processes. A model may initially be configured with a set of parameters and architectural structures that define its behavior, and this configuration may change automatically as the model encounters training inputs, validation data, reference data, or instructor-provided feedback. The model may modify its internal state through optimization techniques, gradient updates, reinforcement signals, vector transformations, attention mechanisms, latent variable adjustments, embedding refinements, or other learning operations executed electronically. Such modifications may occur over extended cycles, partial cycles, or continual learning sequences without explicit intervention by a human.

The disclosure contemplates systems involving data ingestion pipelines that gather input from sources including but not limited to sensor signals, event streams, text data, image data, audio data, video data, structured and unstructured repositories, application logs, telemetric feeds, network services, or human-generated content. Ingestion functions may include filtering, normalization, augmentation, segmentation, batching, tokenization, windowing, compression, encryption, decryption, hashing, deduplication, contextualization, and mapping to internal formats. Intermediate components may transform this data into derived representations, including embeddings, latent encodings, feature tensors, multi-modal joint representations, or contextual vectors suitable for use by downstream modeling engines. These transformations may be performed using neural networks, statistical encoders, dimensionality-reduction algorithms, or hybrid computational modules.

The disclosure contemplates machine learning systems that may employ advanced architectures such as transformer networks, encoder-decoder stacks, mixture-of-experts structures, diffusion models, recurrent networks, convolutional hierarchies, attention-based models, retrieval-augmented architectures, cross-modal alignment engines, graph neural networks, probabilistic models, auto-encoding frameworks, or hybrid symbolic-neural systems. Such models may implement deep layers configured to perform operations including attention calculations, feed-forward projections, gating operations, positional encoding, normalization steps, multi-head routing, sequential decoding, or latent pathway selection. Multi-modal systems may combine textual, visual, auditory, sensory, or structured inputs within joint representational spaces. Embeddings may be learned from large corpora or multi-modal datasets and may encode semantic, syntactic, structural, temporal, spatial, or contextual relationships across modalities. These embeddings may be dynamically updated as the system encounters new information, thereby improving consistency, expressiveness, or alignment with real-world contexts.

The disclosure contemplates training processes that may involve supervised learning, unsupervised learning, semi-supervised learning, self-supervised learning, reinforcement learning, preference optimization, curriculum-based learning, active learning, or continual learning. Training operations may include forward passes through the model, backward propagation of gradients, update steps using optimization algorithms, adaptive learning-rate scheduling, regularization steps, loss-function evaluation, and checkpointing of intermediate states. Training datasets may include real-world data, synthetic data, simulated data, augmented data, or mixtures thereof. Validation procedures may evaluate performance metrics, generalization behavior, safety constraints, or compliance with domain-specific criteria. In some implementations, refinement cycles may incorporate human-in-the-loop interventions, reward model shaping, safety evaluator feedback, or guided corrections.

The disclosure contemplates distributed or federated execution in which computation is partitioned across multiple hardware devices, regions, or clusters. Certain operations may occur at edge devices for low latency, while others may be delegated to remote servers, cloud clusters, datacenters, or specialized compute fabrics. Components may communicate over wired or wireless networks supporting data exchange, synchronization, replication, or model-state updates. Distributed learning processes may synchronize gradients, coordinate model versions, merge updates across shards, or exchange activation values within parallel training regimes. Distributed inference may involve routing requests across replicas, balancing load through orchestration layers, or selecting model pathways dynamically. Network connections may include encryption, authentication, secure session management, or routing protocols appropriate for maintaining privacy, integrity, or availability.

The disclosure contemplates orchestration layers capable of managing complex workflows involving model invocation, tool invocation, external data retrieval, decision routing, fallback selection, multi-model aggregation, post-processing evaluation, or safety governance. Orchestration environments may evaluate contextual signals, metadata, user characteristics, or policy constraints to determine which models, subsystems, or computational branches should be executed. Such environments may dynamically alter execution pathways based on estimated performance, resource availability, model confidence, safety risk, or real-time system health. Post-processing components may evaluate generated outputs for compliance with content policies, statutory requirements, operational constraints, or domain-specific decision rules.

The disclosure contemplates safety-oriented components that evaluate model outputs or intermediate representations for consistency with safety criteria, quality thresholds, regulatory considerations, factual accuracy constraints, domain restrictions, or alignment requirements. Safety modules may employ auxiliary models, discriminators, rule sets, statistical detectors, confidence estimators, or hybrid evaluators to identify undesirable outputs. These modules may trigger remediation actions, including output modification, output rejection, re-routing through alternate inference pathways, invocation of corrective models, or escalation for human review. Safety processes may incorporate real-time validation, contextual scoring, adversarial robustness analysis, anomaly detection, or controlled generation constraints.

The disclosure contemplates governance structures including policy managers, audit loggers, compliance trackers, version controllers, and provenance systems that associate model outputs with contextual metadata, historical signals, update events, training sources, or safety evaluations. Systems may maintain lineage records documenting which model version, configuration state, or training dataset contributed to an outcome. Governance modules may ensure that system behavior aligns with formal requirements such as fairness principles, legal obligations, industry standards, or institutional guidelines.

The disclosure contemplates storage and memory systems capable of storing model parameters, datasets, embeddings, logs, metrics, checkpoints, execution traces, and auxiliary information used to configure or interpret model behavior. The computing device may include these storage systems. Storage media may contain instructions, configurations, or data structures that, when accessed by the computing device, configure that device to carry out the operations described herein. Such media may include executables, bytecode, machine code, firmware, microcode, program modules, configuration files, architectural descriptors, or schema definitions.

The disclosure contemplates user interfaces that permit human operators to view model outputs, initiate tasks, modify configurations, inspect metrics, interact with logs, evaluate safety signals, or guide system adaptation. Interfaces may be multimodal and may support textual input, speech commands, visual interaction, gesture control, or programmatic invocation through application programming interfaces (APIs). Administrative interfaces may allow for reviewing system performance, tuning operational thresholds, enabling or disabling features, monitoring resource use, examining generated content, or initiating refinement workflows.

The disclosure contemplates systems in which instructions are executed entirely on a single device, partially on multiple devices, or cooperatively across remote and local environments. Code may execute directly on hardware, within firmware, inside virtual machines, inside containers, or through any combination of software and hardware interactions. Computational instructions may be stored locally, transferred via communication networks, or streamed from remote systems.

Interpretation of terms in this disclosure is governed by principles commonly applied by persons of ordinary skill in the relevant field. Technical and scientific terms used herein should be understood in a manner consistent with their usage in the field of artificial intelligence, machine learning, computing, networking, data storage, or any related discipline. Terms describing functionality should not be interpreted as strictly structural unless explicitly stated. Phrases such as configured to, adapted to, operable to, or capable of, indicate permissible functionality rather than structural limitations. Terms such as a or an encompass one or more unless clearly contradicted by context. Terms joined by or should be interpreted as inclusive, and terms joined by and should be interpreted as collective.

The description set forth herein provides a broad and flexible framework intended to support a wide range of computer-implemented, machine-learning-enabled, distributed, and multimodal embodiments. Variations may include reallocation of tasks, substitution of algorithms, reconfiguration of models, changes to pipeline ordering, or adoption of alternate hardware. No combination or arrangement mentioned herein should be regarded as required unless explicitly stated.

Further, the disclosure may provide a distributed or cloud-based operation which may include multiple physical or virtual instances of computing devices, distributed across data centers or network boundaries. Functions may be partitioned across machines to achieve parallelism, redundancy, fault tolerance, or improved throughput. Distributed systems may use load balancing mechanisms to maintain stable processing, memory, or bandwidth utilization across clusters and avoid overload conditions. Such deployments may require communication over wired or wireless networks that implement a variety of protocols, including HTTP, HTTPS, MQTT, CoAP, or any other suitable communication framework. Communication channels may include local networks, wide-area networks, personal-area networks, or global communication systems, potentially utilizing secure, encrypted sessions such as SSL-based channels.

Further, the disclosure may provide an algorithm, process, or flow diagram that may include operations that may occur in sequences, reversed orders, concurrently, or in partially overlapping timelines, depending on the implementation. Blocks representing actions in a flowchart may correspond to program modules, instruction sequences, or hardware logic capable of performing the specified acts. Such operations may manipulate physical quantities such as electrical or magnetic signals stored or transferred among memory units, registers, storage devices, or communication media. Flow diagrams may be realized through software running on general-purpose processors, through dedicated hardware circuits, or through combinations of both.

Further, the disclosure may provide a user interface which may include graphical displays, dashboards, selection controls, input fields, monitoring elements, or multimodal interaction surfaces, allowing users to interact with computing systems in speech, touch, gesture, or other modalities. Such interfaces may be presented through client devices, server applications, or remote access platforms and may support visualization of model behavior, system performance, or configuration parameters.

Further, the described features may be combined, rearranged, omitted, or substituted without departing from the principles disclosed. Variations may involve distributing functionality across devices, merging components, implementing features in hardware rather than software, or employing alternative communication protocols. Many such variations and modifications are intended to fall within the scope of the disclosure as understood by persons skilled in the art.

The detailed description of the drawings, therefore, provides a foundation for describing technical, architectural, and operational aspects of embodiments, while allowing broad flexibility in how such embodiments may be implemented in practice. The scope of such embodiments is governed by the claims rather than the illustrative content of the drawings.

In some embodiments, a system consistent with this disclosure includes one or more client devices, one or more servers, and one or more data stores coupled by one or more networks. The client devices may include computing platforms equipped with data processing hardware and memory hardware. The servers may include data servers, application servers, web servers, proxy servers, or cloud computing services that provide shared processing, storage, and networking resources. The data stores can include databases, object stores, file systems, or other repositories that persist configuration data, training data, logs, model artifacts, and other information.

The networks can include public and private networks, such as local area networks, wide area networks, and cloud networks, using wired or wireless communication links. The networks can provide routing, addressing, access control, encryption, and related functionality using standard or proprietary protocols.

For purposes of this disclosure, artificial intelligence systems may include arrangements of software and hardware that perform tasks such as perception, prediction, planning, or generation based on input data. These systems can employ one or more models, such as statistical models, neural networks, decision trees, or other machine learning models. As used herein, a “model” can refer to a parameterized function, an ensemble of such functions, or a collection of cooperating components that process data and produce outputs.

In some embodiments, the system includes a data input engine that obtains data from one or more sources, such as application logs, sensor streams, structured databases, and unstructured content. The data input engine can retrieve, filter, aggregate, or transform the data into feature representations suitable for model consumption. Data sources can include training data, validation data, and reference data used to evaluate and calibrate model behavior. Further, the system includes a modeling engine that manages one or more training processes for one or more models. Moreover, the modeling engine can select model architectures, initialize parameters, and apply training algorithms such as supervised learning, semi supervised learning, unsupervised learning, reinforcement learning, or combinations thereof. The modeling engine can also manage hyperparameters, training schedules, and evaluation procedures across epochs or passes through the data. Further, the system may include a generative response engine or an inference engine that receives prompts or other inputs and generates outputs using one or more models. For example, a natural language interface can receive a text prompt, embed or otherwise encode the prompt, process the encoded prompt using a transformer based model or other sequence model, and generate a sequence of tokens that are decoded into an output. The engine can generate multiple candidate outputs and apply validation or ranking logic to select a final result according to quality, safety, or relevance criteria. Further, the system may include a feedback engine that collects explicit or implicit feedback signals, such as user ratings, corrective edits, or outcome metrics derived from downstream tasks. Furthermore, the system may include a refinement engine that uses the feedback to adjust model parameters, routing logic, or policies, for example, by performing additional training steps, updating reward models, or modifying configuration parameters.

The systems described herein can be implemented using centralized, decentralized, or hybrid arrangements. For instance, models may be deployed in cloud environments, on edge devices, or across both, depending on requirements such as latency, privacy, cost, and reliability. Load balancing and resource management components can distribute processing across devices or data centers and can provide elasticity to accommodate changing workloads.

Certain embodiments may expose functionality through application programming interfaces, software development kits, or graphical user interfaces. Client applications can submit requests to backend services, which can apply authentication, authorization, logging, and policy enforcement before invoking models or tools and returning results.

The systems and methods disclosed herein can be implemented in hardware, software, firmware, or any combination thereof. In some embodiments, operations are carried out by one or more processors executing program instructions stored on one or more non-transitory computer readable media. Program instructions, when executed by the processors, cause the processors to perform the operations described herein.

Instructions can be delivered to computing devices in various ways, such as pre-installation, physical distribution of media, or transmission over networks. Instructions received over a network can be stored in memory or persistent storage and then executed by one or more processors. Dedicated hardware logic, such as application-specific integrated circuits or field programmable gate arrays, can be used alone or in combination with software to implement certain functionality.

Any methods described in connection with embodiments of the present disclosure can be represented as one or more flow diagrams or state diagrams. Blocks in such diagrams can correspond to modules, components, operations, or code segments that implement the associated functionality. Blocks can be reordered, combined, executed concurrently, or omitted according to implementation-specific considerations, unless a particular ordering is required by the claims.

Examples and embodiments described herein illustrate, rather than limit, the claimed subject matter. Certain features have been described in connection with particular embodiments for clarity, but other embodiments can include such features in different combinations. Features described in separate embodiments can be combined, and features described in a single embodiment can be separated.

In general, the method(s) disclosed herein may be performed by at least one computing device. Further, the at least one computing device may be and/or may include at least one server (i.e., at least one server computer). Further, the at least one computing device may be and/or may include a communication device, a processing device, and a storage device. For example, in some embodiments, the method may be performed by the at least one server in communication with at least one client device over a communication network such as, for example, the Internet. In some other embodiments, the method may be performed by one or more of at least one server computer, at least one client device, at least one network device, at least one sensor, and at least one actuator. Examples of the at least one client device, and/or the at least one server computer may include, a desktop computer, a laptop computer, a tablet computer, a personal digital assistant, a portable electronic device, a wearable computer, a smartphone, an Internet of Things (IoT) device, a smart electrical appliance, a video game console, a rack server, a super-computer, a mainframe computer, mini-computer, micro-computer, a storage server, an application server (e.g. a mail server, a web server, a real-time communication server, an FTP server, a virtual server, a proxy server, a DNS server, etc.), a quantum computer, and so on.

### Overview

The present disclosure describes an adaptive tattoo machine with real-time sensor-based closed-loop and predictive control and an augmented reality glass-based stencil placement, manipulation, alignment, and guidance system.

The tattooing system includes a handheld tattoo machine integrated with a real-time adaptive control architecture and a wearable augmented reality stencil alignment and guidance system. The tattoo machine includes a motor-driven reciprocating needle assembly, a needle drive mechanism, and a stroke-adjustment actuator that varies stroke length during active operation. One or more sensor systems detect operational and skin-interaction parameters including resistance, torque, vibration, displacement, position, and motion. A control mechanism receives sensor measurements, identifies changes in interaction conditions, and generates control signals that actuate the stroke-adjustment actuator and modify tattooing parameters during operation. All operations occur continuously in a closed-loop configuration. The tattoo device is capable of operating independently of the augmented reality system and artificial intelligence components, wherein the control mechanism performs adaptive adjustment based solely on sensor input and embedded control logic.

Further, in some embodiments, the system includes a wearable augmented reality glass configured to generate, position, display, manipulate, and maintain alignment of a digital stencil relative to a target skin surface. A spatial tracking and mapping system detects anatomical features, skin curvature, deformation, stretching, compression, and subject movement, and continuously updates stencil registration during tattooing. A user interaction interface permits translation, rotation, scaling, mirroring, opacity adjustment, curvature adaptation, anchoring, and recall of stencil configurations.

The system includes a visual analysis mechanism that compares real-time tattoo execution against a reference stencil and generates corrective guidance. The mechanism detects movement, analyzes ink absorption, and identifies tattooing technique based on motion behavior. A control mechanism generates control signals that modify operation of the tattoo device according to detected conditions, including automatic adjustment of parameters corresponding to lining, shading, color packing, or stippling modes.

Further, the disclosed system relates generally to tattooing devices, tattooing systems, and tattoo execution technologies, and more particularly to adaptive tattoo machines, sensor-assisted tattoo control systems, wearable augmented reality stencil systems, motion-responsive machine control, artist-assistance systems, and integrated tattoo operating platforms capable of real-time analysis, guidance, and parameter adjustment during tattooing.

Further, conventional tattoo machines generally require manual selection of stroke length, operating voltage, give, and related performance characteristics before or during use. In practice, proper tattoo execution depends heavily on the artist's experience, hand speed, depth control, skin assessment, and ability to compensate for variations in anatomy, elasticity, hydration, movement, and ink response. Existing stencil approaches typically rely on physical transfer media applied to the skin, which may distort, fade, smear, shift, or fail to account for dynamic movement of the body during the procedure.

Further, existing tattoo equipment also lacks meaningful real-time feedback regarding skin condition, needle interaction, pigment saturation, unsafe operating conditions, or deviation from intended tattoo paths. Likewise, existing systems do not adequately integrate machine control with wearable augmented reality devices capable of maintaining a digital stencil in alignment with a moving and deforming skin surface.

Further, there remains a need for an integrated tattooing system capable of adaptive machine control, dynamic stencil placement and tracking, artist guidance, motion-based technique recognition, safety monitoring, and ecosystem-wide interoperability across tattoo hardware, wearable visualization systems, data platforms, and training systems.

In some embodiments, the present disclosure describes an integrated tattooing platform combining a tattoo machine with adaptive control and an augmented reality visualization and guidance system.

Further, in some embodiments, the present disclosure describes a handheld tattoo machine including a motor-driven reciprocating needle assembly and a dynamically controllable stroke mechanism capable of real-time stroke length adjustment during active operation.

Further, in some embodiments, the present disclosure describes a sensor-assisted control architecture configured to detect tattooing conditions including resistance, torque, displacement, vibration, skin interaction, and ink deposition response, and to regulate operating parameters in real time.

Further, in some embodiments, the present disclosure describes a wearable augmented reality glass configured to display a digital stencil on a target skin surface, permit user manipulation of the stencil, and maintain stencil alignment through spatial mapping and tracking.

Further, in some embodiments, the present disclosure describes a computer vision and artificial intelligence guidance system configured to compare tattoo execution against an intended path and provide real-time corrective cues, warnings, overlays, or control adjustments.

Further, in some embodiments, the present disclosure describes a motion-responsive control system configured to detect hand speed, acceleration, dwell time, and motion patterns of the tattoo machine and to identify or infer a tattooing mode including lining, shading, color packing, or stippling, and automatically match machine parameters to the detected mode.

Further, in some embodiments, the present disclosure describes a skill-file, training, playback, robotic-assist, operating system, and cloud ecosystem for storing, distributing, evaluating, replaying, and improving tattoo techniques across compatible devices.

In some embodiments, the tattooing system includes a handheld tattoo device comprising a motor, a reciprocating needle assembly, and a stroke-adjustment mechanism that varies stroke length during operation.

Further, in some embodiments, the disclosed system includes an electronically controllable stroke-adjustment mechanism comprising an actuator selected from an electromechanical actuator, micro-linear actuator, piezoelectric actuator, magnetic actuator, servo mechanism, or equivalent precision positioning mechanism capable of sub-millimeter adjustment. The actuator varies stroke length of a reciprocating needle assembly during active operation without requiring interruption of the tattooing process. The stroke-adjustment mechanism operates continuously and performs real-time adjustments based on control signals generated during tattoo execution. The actuator may include any mechanism capable of dynamically modifying stroke length during operation.

Further, in some embodiments, the disclosed system includes one or more sensors configured to detect resistance, torque, vibration, displacement, acceleration, orientation, pressure, skin contact, needle excursion, ink deposition, optical characteristics, spectral characteristics, cartridge state, and environmental conditions. Sensor data is fused to determine real-time operating conditions and skin response. In some embodiments, the system includes one or more communication interfaces configured for wired or wireless communication between the tattoo device, augmented reality system, and external computing systems, including encrypted communication protocols. In some embodiments, sensor data is processed using sensor fusion techniques to combine multiple sensor inputs into a unified representation of tattooing conditions. In some embodiments, sensor measurements are continuously updated during operation at a rate sufficient for real-time control.

Further, in some embodiments, a control mechanism executes a control algorithm that receives sensor data, identifies resistance changes and variations in interaction conditions between the tattoo device and a skin surface, and generates control signals that actuate one or more mechanisms to adjust machine parameters including stroke length, frequency, voltage, torque, give, and needle depth. The control mechanism operates continuously without interruption and performs parameter adjustments in real time during needle insertion into the skin surface. In some embodiments, the control mechanism includes predictive logic that identifies impending changes in skin interaction conditions and modifies tattooing parameters prior to such changes occurring at the needle-skin interface.

In some embodiments, the control mechanism performs continuous parameter adjustment during active needle insertion into the skin surface.

As used herein, the terms control mechanism, control system, and tattoo session controller refer to the same control architecture unless explicitly distinguished. The tattoo session controller is an instance of the control mechanism and operates within the same control architecture to coordinate sensing, analysis, and actuation during tattoo execution.

In some embodiments, the system includes a wearable augmented reality device comprising displays, cameras, depth sensors, and inertial sensors. A spatial tracking mechanism maps a target skin surface and maintains alignment of a digital stencil. The augmented reality device operates in synchronization with the tattoo device, wherein stencil alignment updates in real time and influences operation of the tattoo device during execution.

The tattoo device and the wearable augmented reality device operate in a bidirectional feedback configuration in which sensor data generated by the tattoo device influences stencil alignment and stencil positional data generated by the augmented reality device influences operation of the tattoo device in real time during tattoo execution.

Operation of the tattoo device is dependent on real-time stencil alignment data, and stencil alignment is continuously updated based on feedback from the tattoo device during execution.

Further, in some embodiments, the disclosed system may include a digital stencil that may be translated, rotated, scaled, mirrored, warped, curved, opacity-adjusted, layered, segmented, recalled, saved, duplicated, or otherwise manipulated by gesture, voice, touch, physical controls, remote input, or combinations thereof. Further, in some embodiments, stencil manipulation may occur prior to or during active tattooing.

Further, in some embodiments, the disclosed system may anchor the digital stencil to detected anatomical landmarks, body contours, reference markers, fiducials, or mapped skin topography. The stencil may remain aligned notwithstanding body movement, breathing, posture changes, skin stretch, compression, or localized movement caused by tattooing. Further, in some embodiments, stencil alignment is dynamically recalibrated in real time based on detected motion, deformation, and predicted movement of the skin surface. Further, in some embodiments, stencil alignment is maintained using real-time three-dimensional surface mapping of the skin. Further, in some embodiments, stencil alignment persists despite partial occlusion or loss of visual reference.

A guidance mechanism compares actual needle position to a reference stencil and identifies deviation, depth risk, missed areas, or overwork conditions. The guidance mechanism generates a confidence score representing deviation from an intended tattoo path. A control mechanism generates control signals that actuate one or more mechanisms to modify tattooing parameters in real time based on the confidence score. The system provides continuous feedback and maintains execution accuracy relative to the stencil.

Further, in some embodiments, the tattoo machine may include an integrated illumination system positioned adjacent to or near the cartridge, needle region, or working zone. The illumination system may enhance visibility of the needle, cartridge, stencil, and ink deposition and may automatically adjust brightness. Further, in some embodiments, illumination is automatically adjusted based on detected environmental conditions.

The system includes motion sensors that detect hand speed, motion pattern, directional change, and dwell time. A classification mechanism identifies a tattooing technique based on movement characteristics. A control mechanism generates control signals that modify operation of the tattoo device to correspond to the identified technique. Further, in some embodiments, the disclosed system may store tattoo sessions, skill files, user profiles, performance analytics, model updates, and training data locally or remotely. The platform may support semi-autonomous execution, robotic systems, training overlays, certification scoring, and cloud-based distribution of tattoo technique files. Further, in some embodiments, the system supports sharing, licensing, or transferring tattoo execution data across multiple users or devices. Further, in some embodiments, stored data includes performance metrics and execution history.

In some embodiments, a tattooing system may include a handheld tattoo device including a motor that drives a reciprocating needle assembly, a needle drive mechanism that converts motor motion into substantially linear reciprocating motion, and an electronically controllable stroke-adjustment mechanism comprising at least one actuator that dynamically varies stroke length during active operation without interrupting motor function. The system may further include a plurality of sensors that detect operational parameters including resistance, torque, vibration, displacement, and skin interaction characteristics. A control mechanism may be operatively coupled to the plurality of sensors and the stroke-adjustment mechanism and may continuously receive sensor data during tattooing, identify interaction changes, and generate control signals that adjust one or more tattooing parameters including stroke length, frequency, voltage, torque, and penetration depth in real time. The system may further include a wearable augmented reality device comprising at least one display and one or more sensors that generate spatial mapping data of a target skin surface. A synchronization module may maintain alignment of a digital stencil relative to the target skin surface and continuously update stencil position based on detected movement and deformation. The control mechanism may coordinate operation of the tattooing device with the augmented reality device such that tattoo execution is performed in alignment with the digital stencil in real time.

In some embodiments, a tattoo machine may include a motor-driven reciprocating needle assembly, a stroke-adjustment actuator that dynamically varies stroke length during machine operation, and one or more sensors that detect operational parameters and interaction conditions between the tattoo machine and a skin surface. A control mechanism may receive sensor measurements, identify resistance changes and variations in interaction conditions, and generate control signals that actuate the stroke-adjustment actuator and regulate machine operation in real time. The tattoo machine may maintain consistent needle penetration across varying skin conditions through continuous adjustment.

In some embodiments, the present disclosure describes an augmented reality stencil management system. Further, the augmented reality stencil management system may include a stencil generation module configured to generate a digital tattoo stencil. Further, the augmented reality stencil management system may include a surface mapping system configured to detect topography and curvature of a target skin surface. Further, the augmented reality stencil management system may include a manipulation interface configured for translation, rotation, scaling, mirroring, opacity adjustment, and curvature adaptation of the digital tattoo stencil. Further, the augmented reality stencil management system may include an anchoring module configured to lock and maintain position of the digital tattoo stencil relative to the target skin surface. Further, in some embodiments, stencil data is associated with one or more reference coordinate systems.

In some embodiments, the tattooing system may include a handheld tattoo device comprising a motor-driven reciprocating needle assembly and a stroke-adjustment mechanism operatively coupled to the needle assembly. A control mechanism may generate control signals that actuate the stroke-adjustment mechanism to vary stroke length during active operation without interrupting motor function. The control mechanism may further generate control signals that adjust one or more operating parameters including stroke length, frequency, voltage, torque, and penetration depth based on detected operating conditions during tattoo execution.

In some embodiments, the tattoo machine system includes a reciprocating needle assembly, a stroke-adjustment actuator operatively coupled to the needle assembly, and a position sensing mechanism that detects displacement corresponding to stroke movement. A control mechanism receives displacement data and generates control signals that actuate the stroke-adjustment actuator to vary stroke length during active operation. The actuator performs continuous micro-adjustments in real time during tattooing, and the control mechanism maintains consistent operation based on feedback received during needle insertion into the skin surface.

In some embodiments, the present disclosure describes a tattoo system. Further, the tattoo system may include a communication system. Further, the tattoo system may include a wearable display configured to present tattoo-related guidance to a local user. Further, the tattoo system may include a remote computing system configured to provide real-time assistance, annotations, overlays, or instructions to the local user. Further, in some embodiments, the system enables remote assistance, guidance, or monitoring from a secondary user or computing system. Further, in some embodiments, remote interaction includes real-time annotation or guidance overlays.

In some embodiments, the tattooing system includes one or more sensors configured to detect unsafe tattooing conditions and a control mechanism configured to generate control signals that modify operation of the tattoo device in response to detected unsafe conditions. In some embodiments, the control mechanism automatically reduces operating parameters or halts operation upon detection of unsafe conditions.

In some embodiments, the present disclosure describes a method of controlling a tattoo machine comprising detecting operational parameters of the tattoo machine during tattooing using one or more sensors, determining interaction conditions between the tattoo machine and a skin surface based on the detected parameters, and dynamically adjusting one or more machine parameters including stroke length, frequency, voltage, or penetration depth in real time during tattoo execution.

Further, in some embodiments, the control algorithm is configured to predict impending resistance changes before the reciprocating needle assembly encounters a different skin condition. Further, in some embodiments, predictive control includes forecasting of skin resistance and interaction behavior based on prior data.

Further, in some embodiments, the control mechanism predicts impending resistance changes before the reciprocating needle assembly encounters a change in skin condition and generates control signals that adjust tattooing parameters prior to the change occurring at the needle-skin interface.

The control mechanism operates with a response latency suitable for real-time control between receipt of sensor data and generation of control signals and performs continuous real-time adjustment.

As used herein, real-time operation refers to processing and response occurring within a time interval sufficient to influence needle behavior during active insertion into a skin surface, including response intervals in the millisecond range.

The system includes a predictive analysis mechanism that processes sensor data and operational data to determine predicted outcomes including ink deposition and skin response. A control mechanism generates control signals based on predicted outcomes and modifies operation of the tattoo device prior to changes occurring at the needle-skin interface.

Further, in embodiments, stroke adjustments occur while the motor remains active. Further, in some embodiments, stroke adjustments occur continuously during operation without interrupting motor function.

Further, in some embodiments, the control mechanism generates control signals that cause the actuator to perform micro-adjustments to the stroke-adjustment mechanism during operation. The micro-adjustments include incremental changes to stroke length, frequency, and related parameters to maintain consistent tattooing performance under varying skin interaction conditions.

Further, in embodiments, a plurality of sensors operates simultaneously in a fused sensor configuration.

Further, in some embodiments, safety thresholds trigger automatic parameter adjustment.

Further, in some embodiments, the system includes a haptic alert interface configured to notify an operator of a detected unsafe condition.

Further, in some embodiments, the wearable augmented reality glass comprises a head-mounted display.

Further, in some embodiments, alignment of the digital stencil is maintained within a predetermined tolerance relative to the target skin surface.

Further, in some embodiments, the augmented reality guidance includes depth indicators.

Further, in some embodiments, the manipulation interface accepts gesture input.

Further, in some embodiments, the manipulation interface accepts voice input.

Further, in some embodiments, stencil warping conforms to detected skin curvature.

Further, in some embodiments, stencil position data is stored for later recall. Further, in some embodiments, stencil opacity is adjustable.

Further, in some embodiments, biometric authentication is required before operation.

Further, in some embodiments, unauthorized use triggers a lockout state.

Further, in some embodiments, operational data is transmitted to an external device.

Further, in some embodiments, predictive analytics are applied to optimize tattooing parameters.

Further, in some embodiments, communication between system components is encrypted.

Further, in some embodiments, the system may include an illumination system positioned adjacent to a cartridge or needle region.

Further, in some embodiments, illumination output adjusts based on ambient conditions or tattooing conditions.

In some embodiments, the tattooing system includes a wearable augmented reality device including at least one display and one or more imaging sensors. The system includes a computer vision module that continuously captures real-time imagery of a target skin surface. The system includes a control mechanism that detects movement, deformation, stretching, or compression of the target skin surface and generates control data that adjusts a projected digital stencil in real time. The system maintains spatial registration of the digital stencil relative to anatomical features and continuously updates position, scale, curvature, and orientation of the stencil during tattoo execution.

In some embodiments, the tattooing system includes a wearable augmented reality device configured to perform predictive alignment adjustment based on detected motion and deformation of a target skin surface. A processing mechanism predicts future movement of the skin surface and adjusts a projected digital stencil in advance of the predicted movement to maintain alignment during tattoo execution.

In some embodiments, the tattooing system includes one or more optical or spectral sensing mechanisms that analyze ink deposition into a skin surface and detect saturation levels during tattoo execution. A predictive analysis mechanism processes sensor data and determines ink absorption characteristics of the skin surface. A control mechanism receives the sensor data and predictive outputs and generates control signals that adjust tattooing parameters including needle depth, stroke speed, and ink flow rate to maintain consistent ink deposition. The system continuously compensates for variations in skin type, hydration, and elasticity.

In some embodiments, the tattooing system includes a wearable augmented reality device configured to capture visual and spatial data, a tattoo machine configured to receive control signals, and a communication interface coupling the wearable augmented reality device and the tattoo machine. A control mechanism processes visual data and generates control signals for the tattoo machine, and the tattoo machine dynamically adjusts operational parameters including needle depth, stroke frequency, pressure, and related parameters based on real-time visual analysis.

In some embodiments, the tattooing system includes a motion tracking system that detects movement of a subject's body, an artificial intelligence prediction engine that forecasts future motion trajectories, and a compensation module that adjusts stencil alignment in advance of movement and modifies tattoo machine operation to compensate for predicted motion. The tattooing system maintains tattooing accuracy during involuntary or voluntary movement of the subject.

Further, in some embodiments, stencil alignment is anchored to detected anatomical landmarks.

Further, in some embodiments, stencil curvature dynamically conforms to 3D skin topology.

Further, in some embodiments, stencil recalibration occurs at a frequency of at least 30 updates per second.

Further, in some embodiments, deformation mapping includes detection of skin stretching during needle contact.

Further, in some embodiments, the one or more optical or spectral sensors detect pigment density in real time.

Further, in some embodiments, needle depth is adjusted in increments between 0.01 millimeter and 0.10 millimeter.

Further, in some embodiments, the control mechanism dynamically reduces ink delivery rate in response to detected oversaturation conditions based on real-time sensor measurements and predictive analysis of ink absorption behavior.

Further, in some embodiments, the control mechanism generates control signals that reduce ink flow upon detection of over-saturation conditions during tattoo execution.

Further, in some embodiments, the control mechanism generates control signals that increase ink deposition upon detection of insufficient saturation during tattoo execution.

Further, in some embodiments, the visual guidance overlays include directional arrows indicating stroke path.

Further, in some embodiments, expert tattoo patterns are overlaid for replication.

Further, in some embodiments, shading gradients are visually rendered in augmented reality before execution.

Further, in some embodiments, the visual guidance overlays include depth warnings based on predicted skin damage risk.

Further, in some embodiments, the tattooing system displays a real-time confidence score for each stroke.

Further, in some embodiments, communication between the wearable augmented reality glasses device and the tattoo machine is wireless and encrypted.

Further, in some embodiments, latency between the wearable augmented reality glasses device and the tattoo machine is maintained within a range suitable for real-time coordinated operation.

Further, in some embodiments, the wearable augmented reality glasses device authenticates the tattoo machine via biometric verification.

Further, in some embodiments, the control instructions include real-time stroke path adjustments.

Further, in some embodiments, motion compensation accounts for breathing-induced movement.

Further, in some embodiments, tremor filtering is applied to stabilize tattoo output.

Further, in some embodiments, predictive models are trained on prior movement data.

Further, in some embodiments, the tattooing system further comprises a cloud-based tattoo model repository.

Further, in some embodiments, tattoo sessions are recorded and replayable in augmented reality.

Further, in some embodiments, artificial intelligence generates suggested improvements to a tattoo design in real time.

Further, in some embodiments, the tattooing system provides safety alerts based on predicted skin trauma thresholds.

Further, in some embodiments, remote experts provide live augmented reality guidance overlays.

In some embodiments, the present disclosure describes a non-transitory computer-readable medium storing a tattoo skill file. Further, the tattoo skill file may include motion trajectory data defining one or more paths of a tattooing instrument. Further, the tattoo skill file may include temporal execution data defining timing, sequencing, and speed associated with the one or more paths. Further, the tattoo skill file may include force and interaction data defining pressure, resistance, and skin-contact parameters. Further, the tattoo skill file may include needle configuration data including depth, stroke length, and frequency. Further, the tattoo skill file may include ink deposition parameters including flow rate, saturation thresholds, and layering instructions. Further, the tattoo skill file may include contextual metadata including skin type, anatomical location, and environmental conditions. Further, the tattoo skill file may include adaptive correction rules configured to modify execution based on real-time feedback. Further, the tattoo skill file is configured to be interpreted by a tattoo device, augmented reality system, or robotic execution system to reproduce a tattooing process.

In some embodiments, the present disclosure describes a tattooing system comprising a runtime execution engine configured to read a tattoo skill file and interpret motion trajectory, timing, and force data. The runtime execution engine generates control signals for a tattoo device or robotic system, and a feedback system compares real-time execution against the tattoo skill file. An adaptive correction module dynamically modifies execution based on detected deviations during operation.

In some embodiments, the present disclosure describes a system. Further, the system may include a tattoo skill file formatted in a platform-independent structure. Further, the system may include a translation engine configured to convert the tattoo skill file into machine-specific control instructions for multiple tattoo devices or robotic systems. Further, the same tattoo skill file is executable across heterogeneous hardware platforms.

Further, in some embodiments, the tattoo skill file further includes multi-layer shading instructions.

Further, in some embodiments, the tattoo skill file encodes color blending sequences.

Further, in some embodiments, execution includes real-time deviation scoring.

Further, in some embodiments, execution is paused upon exceeding safety thresholds.

Further, in some embodiments, translation accounts for device-specific mechanical tolerances.

Further, in some embodiments, the tattoo skill file is encrypted to prevent unauthorized use.

Further, in some embodiments, the tattoo skill file is stored in a cloud-accessible repository.

In some embodiments, the tattooing system includes a robotic or motor-assisted tattoo device, a control mechanism that executes predefined tattoo instructions, a human override interface allowing an operator to guide or intervene in execution, and a sensor system that monitors skin interaction. The control mechanism adjusts execution in real time and the system operates in a semi-autonomous mode combining automated execution with human supervision.

In some embodiments, the tattooing system includes a robotic arm or automated positioning system that controls a tattooing instrument, a computer vision system that tracks a target skin surface, an artificial intelligence controller that generates tattoo execution paths and controls motion, pressure, and ink deposition, and a safety system that detects unsafe conditions and halts operation. The tattooing system performs tattooing with reduced continuous human intervention.

In some embodiments, the present disclosure describes a tattooing system. Further, the tattooing system may include a tattoo device operated by a human artist. Further, the tattooing system may include an artificial intelligence system configured to assist in motion guidance, provide real-time correction, and assume partial control of execution upon detecting deviation. Further, control dynamically shifts between the human artist and the artificial intelligence system during tattooing.

Further, in some embodiments, automation includes line tracing.

Further, in some embodiments, automation includes shading execution.

Further, in some embodiments, the robotic arm or automated positioning system compensates for skin movement.

Further, in some embodiments, execution accuracy is maintained within sub-millimeter tolerance.

Further, in some embodiments, artificial intelligence takeover is triggered by deviation thresholds.

Further, in some embodiments, biometric monitoring of the subject is performed during tattooing.

Further, in some embodiments, execution logs are recorded for playback and analysis.

In some embodiments, the present disclosure describes a training system. Further, the training system may include a wearable augmented reality glasses device. Further, the training system may include a training module configured to display guided tattoo execution overlays. Further, the training system may include a performance tracking system configured to monitor user actions. Further, the training system may include an evaluation engine configured to score performance based on accuracy, timing, technique, or combinations thereof. Further, the training system trains a user to perform tattooing procedures using augmented reality guidance.

In some embodiments, the present disclosure describes a system. Further, the system may include a database of tattoo skill benchmarks. Further, the system may include an artificial intelligence evaluation engine configured to analyze recorded tattoo execution data and compare performance to benchmark standards. Further, the system may include a certification module configured to generate a credential indicating skill level. Further, the credential is based on objective performance metrics.

In some embodiments, the present disclosure describes a system. Further, the system may include a cloud platform configured to store tattoo skill files. Further, the system may include a distribution system configured to allow users to access, purchase, or license tattoo skill files. Further, the system may include a compatibility engine configured to enable playback across multiple devices. Further, tattoo techniques are distributed as digital assets.

Further, in some embodiments, training includes real-time corrective overlays.

Further, in some embodiments, simulated practice is performed on virtual or synthetic skin models.

Further, in some embodiments, certification levels correspond to complexity tiers.

Further, in some embodiments, certification is stored on a blockchain ledger.

Further, in some embodiments, creators receive royalties from tattoo skill file usage.

Further, in some embodiments, tattoo skill files include ratings and performance analytics.

Further, in some embodiments, historical performance data is used to personalize training.

In some embodiments, the present disclosure describes a tattoo operating system. Further, the tattoo operating system may include a centralized control architecture configured to coordinate operation of a tattoo device, augmented reality glasses, and one or more sensor systems. Further, the tattoo operating system may include a real-time data processing engine configured to receive and process motion data, skin interaction data, and visual data. Further, the tattoo operating system may include a modular software framework comprising a stencil rendering module, a motion control module, a skin analysis module, and an artificial intelligence decision engine. Further, the tattoo operating system may include a synchronization layer configured to maintain alignment between physical execution and digital representations. Further, the tattoo operating system orchestrates tattoo execution, visualization, and adaptation in real time.

Further, in some embodiments, the tattoo operating system may include an application programming interface configured to allow third-party modules to interact with the tattoo operating system. Further, the tattoo operating system may include a plugin architecture configured to enable installation of software modules including design tools, training systems, and execution enhancements. Further, the tattoo operating system functions as a platform for extensible tattoo-related applications.

In some embodiments, the present disclosure describes a system. Further, the system may include a network of tattoo operating systems. Further, the system may include a cloud infrastructure configured to synchronize data across devices. Further, the system may include a shared intelligence layer configured to update models based on aggregated data. Further, improvements learned from one system propagate across the network.

Further, in some embodiments, the tattoo operating system supports real-time firmware updates.

Further, in some embodiments, software modules operate independently within a sandboxed architecture.

Further, in some embodiments, applications include monetized software extensions.

Further, in some embodiments, anonymized tattoo data is aggregated for model training.

Further, in some embodiments, user profiles store personalized tattoo settings.

Further, in some embodiments, access is controlled via authentication credentials.

In some embodiments, the present disclosure describes a system. Further, the system may include a 3D scanning module configured to generate a digital model of a subject's skin surface. Further, the system may include a simulation engine configured to create a digital twin representing physical or biological properties of the skin surface. Further, the system may include a prediction module configured to simulate tattoo outcomes including ink spread, color blending, or healing effects. Further, the digital twin enables pre-execution visualization and optimization.

In some embodiments, the tattooing system includes a digital twin of a target skin surface representing physical, geometric, and biological characteristics of the skin. The system further includes a real-time data input mechanism that updates the digital twin during tattooing based on sensor data, visual data, and motion data. The system further includes a predictive model that forecasts ink dispersion, tissue response, deformation behavior, and healing characteristics. A control mechanism dynamically adjusts tattooing parameters including needle depth, stroke length, frequency, voltage, torque, and ink flow rate based on predictions generated by the predictive model and real-time feedback from the digital twin. The system continuously updates the digital twin and modifies tattooing parameters during execution.

The tattooing system includes a digital twin of a target skin surface representing physical, geometric, and biological characteristics. A real-time update mechanism modifies the digital twin during tattooing based on sensor data and visual data. A predictive model determines ink dispersion and tissue response. A control mechanism generates control signals that adjust tattooing parameters including needle depth, stroke length, frequency, voltage, and torque based on the digital twin and predicted behavior.

Further, in some embodiments, the digital twin includes elasticity modeling.

Further, in some embodiments, hydration levels are simulated.

Further, in some embodiments, simulation updates occur in real time.

Further, in some embodiments, predictions include scar formation risk.

Further, in some embodiments, anatomical curvature is modeled.

Further, in some embodiments, simulation output informs augmented reality overlay adjustments.

In some embodiments, the tattooing system includes one or more sensors that detect biological characteristics of skin including thickness, hydration, vascularity, pigmentation, or combinations thereof. An analysis mechanism classifies skin condition, and a safety mechanism determines safe tattooing parameters. The system prevents or reduces damage by adapting to detected biological conditions.

In some embodiments, the present disclosure describes a system. Further, the system may include a detection module configured to identify conditions including inflammation, irritation, abnormal skin responses, or combinations thereof. Further, the system may include an artificial intelligence model configured to predict adverse outcomes. Further, the system may include a control mechanism configured to adjust or halt tattooing.

In some embodiments, the present disclosure describes a system. Further, the system may include a data interface configured to integrate with medical records or dermatological databases. Further, the system may include an analysis engine configured to incorporate clinical data into tattoo planning. Further, the tattooing decisions are informed by medical-grade information.

Further, in some embodiments, analysis includes melanin density detection.

Further, in some embodiments, vascular mapping is performed.

Further, in some embodiments, alerts are generated prior to skin damage.

Further, in some embodiments, machine operation is automatically halted upon detection of a threshold risk.

Further, in some embodiments, prior skin conditions are considered.

Further, in some embodiments, safe depth ranges are dynamically calculated.

Further, in some embodiments, recommendations are provided to an operator.

In some embodiments, the present disclosure describes a system. Further, the system may include a tattoo operating system. Further, the system may include a digital skill file format. Further, the system may include an augmented reality guidance system. Further, the system may include a robotic or semi-autonomous execution system. Further, the system may include a cloud-based distribution platform. Further, in some embodiments, the tattoo operating system, the digital skill file format, the augmented reality guidance system, the robotic or semi-autonomous execution system, and the cloud-based distribution platform operate within an integrated ecosystem.

Further, in some embodiments, the integrated ecosystem requires licensing.

Further, in some embodiments, usage data is tracked for monetization.

Further, in some embodiments, third-party developers create extensions compatible with the integrated ecosystem.

Further, in some embodiments, updates are deployed over the air.

Further, in some embodiments, security includes encryption protocols.

Further, in some embodiments, user identity is authenticated biometrically.

Further, in some embodiments, analytics dashboards are provided.

Further, in some embodiments, marketplace transactions are supported.

Further, in some embodiments, artificial intelligence continuously improves system performance.

In some embodiments, the tattoo machine includes a housing configured to be held by a user, a motor-driven reciprocating needle assembly, one or more motion sensors that detect movement of the tattoo machine during operation, and a control mechanism that analyzes movement characteristics including speed, acceleration, directional change, and dwell time. The control mechanism generates control signals that automatically adjust tattooing parameters including stroke length, needle frequency, motor speed, torque, penetration depth, and ink delivery characteristics based on detected movement to match a corresponding tattooing technique.

Further, in some embodiments, the detected or inferred tattooing technique comprises lining.

Further, in some embodiments, the detected or inferred tattooing technique comprises shading.

Further, in some embodiments, the detected or inferred tattooing technique comprises color packing.

Further, in some embodiments, the detected or inferred tattooing technique comprises stippling.

Further, in some embodiments, slower hand speed causes the control module to increase stroke length or penetration consistency for lining.

Further, in some embodiments, sweeping hand motion causes the control module to reduce stroke aggressiveness for shading.

Further, in some embodiments, slower high-contact movement causes the control module to increase saturation-oriented settings for color packing.

Further, in some embodiments, intermittent tapping movement causes the control module to shift to stippling settings.

Further, in some embodiments, the control mechanism classifies tattooing technique based on movement data including speed, acceleration, directional change, and dwell time. The control mechanism generates control signals that modify operation of the tattoo device according to the classified technique including lining, shading, color packing, or stippling.

Further, in some embodiments, the control module transitions between technique settings without stopping the tattoo machine.

Further, in some embodiments, the one or more motion sensors comprise one or more accelerometers, gyroscopes, inertial measurement units, or combinations thereof.

The control mechanism generates a confidence score corresponding to an inferred tattooing technique based on detected motion data and sensor input. The control mechanism dynamically modifies one or more tattooing parameters in real time based on the confidence score.

Further, in some embodiments, the control mechanism combines hand speed data with skin resistance data to determine interaction conditions between the tattoo device and a target skin surface. The control mechanism generates control signals that modify one or more tattooing parameters including stroke length, frequency, voltage, torque, and penetration depth based on the combined data.

In some embodiments, the tattoo session controller comprises one or more electronic control circuits configured to coordinate acquisition of tattoo-related data, real-time analysis of the acquired data, and generation of control signals that actuate one or more tattooing devices, visualization devices, and sensor devices to perform continuous tattoo executions.

In some embodiments, operation data includes control signals, sensor measurements, stencil alignment data, motion data, deformation data, and feedback data used by the control mechanism to coordinate tattoo execution, stencil alignment, and parameter adjustment continuously.

In one embodiment, an operation data record includes: (i) a session identifier, (ii) a device identifier, (iii) a timestamp, (iv) a coordinate frame identifier, (v) a command type, (vi) a payload length, (vii) a payload containing parameter values or display primitives, (viii) a safety flag, (ix) a confidence score, and (x) a checksum or message authentication code.

In some embodiments, a skin interaction parameter may include any computed value representing interaction between a tattooing device and skin, including contact pressure, normal force, lateral force, needle penetration estimate, tissue resistance, vibration response, elastic rebound, contact stability, skin drag, needle load, ink uptake, tissue trauma index, or combinations thereof.

In some embodiments, a stencil data may include vector outlines, raster images, layered design objects, control points, Bezier curves, depth values, mesh vertices, texture maps, scale factors, rotation values, opacity values, color values, anatomical landmark associations, fiducial associations, or surface registration parameters.

In some embodiments, an alignment data may include one or more transformations mapping a stencil coordinate frame to a skin coordinate frame, including translation, rotation, scale, affine transform, projective transform, non-rigid deformation field, mesh-to-mesh correspondence, or combinations thereof.

In some embodiments, an anchoring data may include a set of anchor points, anchor descriptors, landmark identifiers, fiducial marker identifiers, confidence values, and constraints used to maintain a digital stencil in a positional relationship with a target skin surface.

Further, in some embodiments, unsafe tattooing condition means a detected or predicted tattooing condition satisfying one or more stored safety rules, including a measured or estimated parameter exceeding or falling below a predetermined threshold, a parameter remaining outside a permitted range for more than a time window, a rapid trend indicating impending unsafe operation, or a classifier output exceeding a risk confidence threshold.

In some embodiments, the tattoo session controller performs the following algorithm:

Initiate a tattoo session and assign a session identifier.

Register one or more devices, including a tattooing device, AR glasses, sensor device, user device, or robotic device.

Receive calibration data identifying device coordinate frames, sensor offsets, actuator limits, display calibration values, and user-selected tattoo design data. Obtain tattoo-related information, including one or more of tattoo design data, skin image data, depth data, tattoo machine sensor data, machine position data, user input data, environmental data, and client-specific skin data.

Time-synchronize the obtained information using timestamps or clock synchronization messages.

Filter sensor data to reduce noise using a low-pass filter, median filter, Kalman filter, complementary filter, particle filter, or moving average filter.

Extract features from the filtered data, including pressure, acceleration, orientation, contact state, surface curvature, anatomical landmarks, fiducial positions, optical intensity, spectral response, path position, speed, dwell time, vibration magnitude, and resistance estimate.

Determine a current tattooing state, including at least one of idle, calibration, stencil placement, lining, shading, color packing, stippling, safety hold, training, guidance, or robotic execution.

Apply rule-based logic, a control model, or a trained machine learning model to determine one or more operations corresponding to the current state.

Generate operation data for the one or more devices, including machine control commands, AR overlay commands, guidance cues, safety alerts, actuator commands, and data logging records.

Validate operation data against safety constraints, device limits, latency constraints, and user permissions.

Transmit validated operation data to the corresponding device.

Receive feedback data from the corresponding device.

Compare feedback data to the commanded operation data.

Update session state and repeat the foregoing steps until the tattoo session is terminated.

Further, in the given manner, the processor is not merely performing an abstract data processing function, but instead performs a tattoo-specific closed-loop control and visualization process tied to tattooing hardware, sensors, AR displays, and tattoo machine actuators.

In some embodiments, the tattooing device includes a housing, a grip portion, a motor, a motor shaft, a motion conversion assembly, a needle bar or cartridge interface, a reciprocating needle assembly, a sensor board, an actuator driver circuit, and a communication interface. The motor may be a brushless DC motor, brushed DC motor, coreless motor, stepper motor, linear motor, or voice-coil actuator.

Further, in some embodiments, the needle drive mechanism may convert rotational motor motion into substantially linear needle motion using an eccentric cam, crank-slider assembly, Scotch yoke, cam follower, linear guide, magnetic coupling, or other motion conversion structure. The needle drive mechanism may include a needle travel axis and one or more bearings, bushings, guide rails, flexures, or low-friction sleeves to maintain needle alignment.

In some embodiments, a stroke-adjustment mechanism includes a servo-driven eccentric cam assembly. A rotatable eccentric member is coupled between the motor shaft and a needle drive linkage. A micro-servo or stepper motor rotates or translates the eccentric member to change an offset distance between the motor axis and the linkage axis. The offset distance determines the stroke length. During operation, the control processor commands the micro-servo to change the offset distance within a permitted range, thereby varying the stroke length without stopping the motor.

In some embodiments, a stroke-adjustment mechanism includes a movable slider carriage supporting a cam follower. A linear actuator, solenoid, piezoelectric actuator, voice-coil actuator, or miniature lead-screw actuator moves the slider carriage along an adjustment axis. Movement of the slider carriage changes the effective lever arm of the motion conversion assembly and varies the travel distance of the needle along the needle travel axis.

In some embodiments, the stroke-adjustment mechanism includes a piezoelectric micro-positioner disposed between the drive linkage and the needle interface. The piezoelectric micro-positioner performs fine stroke-length adjustment or needle-depth trim while a larger mechanical adjustment mechanism provides coarse adjustment.

Further, in some embodiments, the stroke length may be adjustable between about 1.0 mm and about 6.0 mm, including between about 2.0 mm and about 5.0 mm. Fine adjustments may be performed in increments of about 0.01 mm to about 0.10 mm. The controller may limit a rate of stroke-length change to avoid abrupt needle movement, for example limiting adjustment to less than 0.2 mm per control interval or less than 1.0 mm per second.

In some embodiments, the tattooing device may include one or more pressure sensors, force sensors, torque sensors, motor current sensors, Hall-effect sensors, encoders, optical displacement sensors, accelerometers, gyroscopes, inertial measurement units, microphones, vibration sensors, thermal sensors, spectral sensors, and skin-contact sensors. The sensors may be located in the grip, motor assembly, cartridge interface, needle drive assembly, housing, or external wearable device.

In some embodiments, the processor identifies skin interaction parameters using the following algorithm:
Receive raw force data from a force sensor near the cartridge interface.
Receive motor current or torque data from a motor driver.
Receive vibration data from an accelerometer or vibration sensor.
Receive needle displacement data from an optical encoder, Hall sensor, or displacement sensor.
Filter each data stream and remove baseline offsets obtained during calibration.
Determine contact pressure from the force data and a known contact geometry.
Determine tissue resistance from a combination of motor current change, needle deceleration, and force data.
Determine contact stability from variance in pressure, acceleration, and displacement over a sliding time window.
Determine a penetration estimate by comparing commanded needle displacement with detected tissue resistance and cartridge geometry.
Determine a skin elasticity estimate from rebound behavior after needle withdrawal or from a pressure-displacement curve.
Generate a skin interaction parameter vector containing the pressure, resistance, contact stability, penetration estimate, elasticity estimate, and confidence values.

Further, the processor may calculate a normalized resistance value R_norm according to:

$$R\_norm = w 1 * normalize ( motor\_current \_delta ) + w 2 * normalize ( force\_normal ) + w 3 * normalize ( needle\_deceleration ) + w 4 * normalize ( vibration\_magnitude )$$

where, w1 through w4 are stored weighting coefficients determined during calibration or training. Further, the normalized resistance value may be mapped to control data according to a rule table, regression model, lookup table, or model predictive controller.

In some embodiments, the processor generates control data according to the following closed-loop control algorithm:
Determine a target tattooing mode selected by a user or inferred from motion data.
Retrieve a base parameter set for the target mode, including base stroke length, base motor speed, base voltage, base torque, base give, base frequency, and base needle depth.
Determine skin interaction parameters from sensor data.
Compare each skin interaction parameter to a desired range for the target mode.
Generate a parameter adjustment vector using one or more of a rule-based controller, PID controller, adaptive controller, or model predictive controller.
Apply safety clamping to ensure adjusted parameters remain within device and skin-safety limits.
Transmit control data to motor drivers, actuator drivers, haptic output devices, displays, or user devices.
Confirm that measured output follows the commanded parameters.
If measured output deviates beyond a tolerance, generate a correction command or alert.

Further, for example, when contact pressure is high and vibration response indicates increased tissue resistance, the controller may reduce motor speed, reduce needle depth, reduce stroke aggressiveness, increase give, or generate a visual warning. When contact pressure is low and ink saturation is insufficient, the controller may increase dwell guidance, increase stroke length within a safe range, increase frequency, or request operator confirmation.

In some embodiments, control data includes a parameter record containing: stroke_length_mm, needle_frequency_hz, motor_voltage_v, torque_limit, give_setting, needle_depth_mm, mode_id, ramp_rate, validity_duration_ms, and safety_state.

In some embodiments, the tattooing device includes an inertial measurement unit configured to generate acceleration and angular velocity data. The processor determines hand speed and technique as follows:
Receive accelerometer, gyroscope, and optional optical tracking data from the tattooing device.
Transform motion data into a tattoo machine coordinate frame or skin coordinate frame.
Estimate tip position, velocity, acceleration, angular velocity, dwell time, directional change frequency, and path curvature.
Segment motion into windows of 100 ms to 2000 ms.
Extract features including average speed, speed variance, stroke periodicity, dwell time, sweep amplitude, path linearity, local curvature, pressure correlation, and contact duration.
Classify the current technique as lining, shading, color packing, stippling, or unknown using a rule set or trained classifier.
Generate a confidence score for the classification.
If the confidence score exceeds a threshold, transition the tattooing device toward a corresponding parameter profile using a ramped transition.
If the confidence score is below the threshold, maintain current settings or request confirmation.

Further, a rule-based classifier may identify lining when path linearity is high, lateral speed is within a lining range, dwell time is low, and pressure is stable. The classifier may identify shading when the motion includes repeated sweeping arcs with moderate speed and low-to-medium pressure. The classifier may identify color packing when hand speed is low, dwell time is high, and saturation feedback indicates fill behavior. The classifier may identify stippling when the motion includes intermittent contact events, high local acceleration changes, and short dwell pulses.

Further, a machine learning classifier may include a random forest, support vector machine, temporal convolutional neural network, recurrent neural network, long short-term memory network, transformer sequence model, or ensemble model trained on labeled motion windows from tattoo artists.

In some embodiments, the wearable visualization device includes a head-worn display, one or more cameras, one or more depth sensors, an inertial measurement unit, a processor, memory, and a wireless communication interface. The visualization device may operate alone or with a separate computing device.

Further, the system may maintain the following coordinate frames:
C—camera coordinate frame of the AR glasses.
W—world coordinate frame maintained by simultaneous localization and mapping.
S—skin surface coordinate frame defined by anatomical landmarks, fiducials, or skin topography.
D—digital stencil coordinate frame.
T—tattoo machine tip coordinate frame.
U—user display coordinate frame.

Further, during setup, the system generates a transformation T_WS from the skin surface coordinate frame to the world coordinate frame and a transformation T_SD from the digital stencil coordinate frame to the skin surface coordinate frame. The display processor generates display data by transforming stencil vertices from D to S, from S to W, and from W to the display coordinate frame.

In some embodiments, stencil registration is performed as follows:
Capture baseline RGB image data and depth data of a target skin surface.
Detect anatomical landmarks, surface contours, pores, freckles, scars, fiducial markers, or temporary reference marks.
Generate a 3D point cloud or mesh representing the target skin surface.
Receive user placement input defining initial stencil position, scale, rotation, and opacity.
Map stencil control points to selected landmarks or mesh points.
Calculate an initial rigid, affine, or non-rigid transform between the stencil and the skin surface.
Store the transform and anchor constraints as alignment data and anchoring data.
Render the digital stencil using the transform so that the stencil appears aligned with the target skin surface.

Further, in some embodiments, a synchronization module may be implemented by instructions that perform time alignment, coordinate transformation, and registration update. In one embodiment, the synchronization module performs the following algorithm:
Receive a live frame from an image sensor and a corresponding depth frame from a depth sensor.
Receive inertial data from an inertial measurement unit.
Estimate current pose of the visualization device relative to the world frame.
Detect current positions of skin landmarks or fiducials.
Match current landmarks or fiducials to baseline anchors using descriptor matching or nearest-neighbor matching.
Calculate a current skin transform by minimizing error between baseline anchor positions and current anchor positions.
Update the display transform for the digital stencil.
Send the updated display transform to the display renderer.

Further, in some embodiments, an anchoring module may be implemented by instructions that perform anchor selection, anchor validation, and anchor maintenance. In one embodiment, the anchoring module performs the following algorithm:
Identify candidate anchors from anatomical landmarks, natural skin features, surface contours, fiducials, or tattoo design reference points.
Assign an anchor confidence value based on visibility, feature uniqueness, tracking stability, and spatial distribution.
Select a set of anchors having confidence values above a threshold and distributed around the stencil area.
Store the selected anchors with baseline 2D image positions, 3D surface positions, feature descriptors, and confidence values.
During tattooing, track selected anchors in live sensor data.
Reject outlier anchors based on reprojection error or sudden displacement inconsistent with neighboring anchors.
Update the stencil transform using remaining anchors.
If anchor confidence falls below a threshold, request recalibration, switch to a backup anchor set, or freeze display updates.

In some embodiments, the system compensates for skin deformation as follows:
During a first time period, generate a baseline mesh of the target skin surface from depth data or multi-view images.
Associate digital stencil vertices with corresponding positions on the baseline mesh.
During a second time period, generate a live mesh of the target skin surface.
Match live mesh regions to baseline mesh regions using anatomical landmarks, fiducials, optical flow, surface descriptors, or iterative closest point alignment.
Compute a displacement vector for each matched region.
Generate a deformation field by interpolating displacement vectors across the mesh.
Apply the deformation field to the digital stencil vertices.
Limit deformation using smoothness constraints, maximum stretch constraints, and anchor constraints.
Render an updated stencil that compensates for skin stretching, compression, curvature change, posture change, breathing-induced movement, or localized skin displacement.

Further, the deformation field may be a thin-plate spline transform, piecewise affine mesh warp, B-spline free-form deformation, optical-flow field, finite-element approximation, or learned deformation model. The processor may generate stencil compensation data including translation compensation, rotation compensation, scale compensation, curvature compensation, warping compensation, and registration correction values.

In some embodiments, execution path data includes one or more intended path segments. Each path segment may include a segment identifier, start point, end point, control points, path type, target speed, target pressure, target depth, target dwell time, target saturation, and permissible tolerance.

Further, in some embodiments, the system may determine an actual execution path by tracking the tattoo machine tip using AR glasses cameras, optical markers, electromagnetic tracking, inertial tracking, ultrasonic tracking, machine-integrated sensors, or combinations thereof.

Further, in some embodiments, the processor determines path deviation as follows:
Receive intended execution path data for a tattoo design layer.
Receive actual tip position, orientation, speed, and time data.
Project the actual tip position into the skin surface coordinate frame.
Identify the nearest intended path segment.
Compute lateral deviation as the shortest distance between the actual tip position and the intended path segment.
Compute angular deviation between actual tool orientation or movement direction and intended path direction.
Compute timing deviation between actual progression and expected progression along the path.
Compute depth deviation using sensor-estimated needle depth or pressure-depth correlation.
Determine whether any deviation exceeds a corresponding threshold.
Generate guidance data including a visual cue, directional arrow, color-coded warning, depth warning, missed-area indication, overwork warning, haptic cue, or audio cue.

Further, for example, if lateral deviation exceeds 0.5 mm, the display may render a directional arrow toward the intended path. If dwell time exceeds a threshold at a location and ink saturation is high, the display may render an overwork warning and the tattooing device may reduce aggressiveness or enter a safety hold.

In some embodiments, optical or spectral sensors generate ink saturation data. The sensors may include RGB cameras, near-infrared sensors, multispectral sensors, polarized light sensors, optical coherence sensors, or colorimeters. The processor may compare pre-ink and post-ink images to estimate pigment density, saturation uniformity, local redness, swelling, or reflectance changes.

Further, in some embodiments, the processor may determine saturation abnormality data using the following algorithm:
Capture a baseline image or spectral measurement of the target skin region before ink deposition.
Capture one or more live images or spectral measurements after needle passes.
Normalize live measurements for illumination, viewing angle, skin tone, and camera exposure.
Segment the tattooed region corresponding to a stencil segment.
Compute pigment density, color uniformity, local contrast, and reflectance change.
Compare computed values to target saturation values for the stencil segment.
Classify the region as under-saturated, acceptable, over-saturated, inflamed, or uncertain.
Generate updated operational parameters or guidance cues.

Further, if under-saturation is detected, the processor may recommend a slower hand speed, increased dwell time, increased stroke length within a safe range, increased needle frequency, or an additional pass. If over-saturation, inflammation, or trauma risk is detected, the processor may reduce needle depth, reduce stroke length, reduce motor speed, increase give, issue an alert, or pause operation.

Further, in embodiments using artificial intelligence, the system may include one or more trained models. The disclosure should be understood to include rule-based, statistical, and machine-learning implementations, and no claim should be limited to a neural network unless expressly recited.

Further, the training data may include recorded tattoo sessions containing synchronized sensor data, AR image data, depth data, machine parameter data, tattoo design data, user action data, path tracking data, ink saturation data, skin condition data, and outcome labels. Labels may include technique class, path deviation, safe/unsafe state, skin condition class, saturation class, deformation vector, guidance cue, or parameter adjustment.

Further, the training pipeline may include:
Collecting synchronized multimodal tattoo session records.
Removing or anonymizing personal identifiers.
Segmenting records into time windows or spatial regions.
Generating feature vectors from sensor data, images, depth maps, and machine data.
Assigning labels from expert annotation, measured outcomes, or rule-based heuristics.
Training one or more models using supervised, semi-supervised, self-supervised, reinforcement learning, or transfer learning techniques.
Validating models on held-out sessions.
Storing model parameters, version identifiers, confidence thresholds, and supported operating ranges.
Deploying the trained model to a tattoo machine, AR glasses, mobile device, local workstation, or cloud server.

Further, the inference pipeline may include:
Receiving live sensor data during a tattoo session.
Preprocessing the live sensor data using the same normalization used during training.
Generating a live feature vector or image tensor.
Applying the trained model to generate an output, such as deformation data, technique classification, saturation classification, path quality score, skin condition classification, or parameter recommendation.
Comparing a confidence score to a threshold.
If the confidence score exceeds the threshold, generating operation data based on the model output.
If the confidence score does not exceed the threshold, using a rule-based fallback, requesting user confirmation, or maintaining current settings.

In some embodiments, safe tattooing parameter data may be generated by selecting a base parameter set based on tattooing mode and then modifying the base parameter set based on skin condition classification, anatomical location, sensor feedback, and safety rules. For example, for sensitive skin or high inflammation score, the system may reduce maximum needle depth, reduce permitted stroke length, lower torque limits, and increase alert sensitivity.

In some embodiments, biological characteristic data is sensor-derived data and does not require a medical diagnosis. Biological characteristic data may include estimated skin thickness, hydration-related optical response, pigmentation-related optical response, melanin density estimate, vascularity estimate, redness score, irritation score, inflammation score, sensitivity estimate, or combinations thereof.

Further, in some embodiments, the processor may classify skin condition as follows:
Acquire optical, spectral, depth, thermal, or pressure-response data from the target skin region.
Normalize the acquired data for illumination, sensor distance, viewing angle, and baseline skin tone.
Extract features such as color channels, spectral ratios, surface texture, local temperature, pressure response, and elastic rebound.
Apply a classifier or rule table to determine a skin condition class, such as normal, dry, highly elastic, low elasticity, high pigmentation, high redness, irritated, inflamed, scarred, thin-skin region, or uncertain.
Generate a confidence value for the classification.
Generate safe tattooing parameter data based on the class and confidence value.
If the confidence value is below a threshold, request user assessment or use conservative default parameters.

In some embodiments, the system generates a digital twin of a target skin region by combining a 3D surface mesh with one or more estimated skin property maps. The skin property maps may represent curvature, elasticity, pigmentation, hydration, texture, thickness-related estimate, and expected ink response.

Further, in some embodiments, the digital twin may be generated as follows:
Capture RGB images and depth data of the target skin region.
Generate a 3D surface mesh having vertices and surface normal.
Map image texture data onto the mesh.
Estimate skin property values for mesh regions using sensor data or user input.
Associate stencil data with the mesh.
Simulate a tattoo outcome by projecting stencil layers onto the mesh and applying color blending, opacity, ink spread, curvature distortion, and optional healing/fading models.
Generate pre-execution visualization data for the AR glasses or user device.

Further, a simplified ink spread model may apply a diffusion kernel to simulated pigment placement, where the kernel width is selected based on estimated skin texture, hydration, and anatomical location. A color blending model may combine pigment color and baseline skin color using opacity and reflectance parameters. A healing model may be trained using prior tattoo outcome images or may use a rule-based fading factor selected by pigment color, skin tone, and anatomical region.

In some embodiments, a tattoo skill file is stored as a structured data object containing:
Header: file version, creator identifier, creation date, supported device classes, checksum.
Design section: vector layers, raster textures, color palette, path segments, layer order.
Motion section: path points, time stamps, speeds, accelerations, dwell times, and orientation targets.
Force section: target pressure, resistance range, contact state, and force tolerances.
Needle section: stroke length, needle depth, needle frequency, needle configuration, and cartridge type.
Ink section: pigment identifier, saturation target, fill strategy, layering order, and blending rules.
Context section: anatomical location, skin type estimate, surface mesh, calibration data, environmental conditions.
Adaptation section: rules for modifying execution based on sensor feedback.
Safety section: thresholds, stop conditions, and user override requirements.
Security section: encryption state, authorization token, license identifier, or digital signature.

Further, in some embodiments, a playback engine may parse the skill file, validate device compatibility, interpolate path data into time-indexed commands, apply device-specific translation, apply safety limits, generate tattoo device control data, generate AR overlay data, and monitor feedback to modify or pause execution.

In some embodiments, a robotic tattooing system includes a robotic arm, end effector, tattooing instrument, force sensor, depth sensor, emergency stop, human override input, computer vision system, and safety controller. The robotic controller maintains a tool center point relative to the skin surface coordinate frame and limits force, speed, and depth.

Further, before robotic execution, the system may perform: (i) surface scan, (ii) stencil registration, (iii) path planning, (iv) collision checking, (v) force calibration, (vi) user authorization, and (vii) dry-run visualization. During execution, the system may monitor skin movement, force, depth, path deviation, saturation, and user override input. If any safety threshold is exceeded, the system may retract the needle, pause motion, disable the motor, or request user intervention.

Further, semi-autonomous embodiments may automate line tracing, shading, or color packing while permitting the human operator to guide the device or override motion. Fully autonomous embodiments may execute path segments without continuous human motion input but may require human supervision and emergency override.

In some embodiments, the training system generates performance parameter data including path accuracy, timing accuracy, pressure consistency, depth consistency, speed smoothness, motion smoothness, dwell time, coverage, missed-area percentage, overwork risk, and completion percentage.

Further, in some embodiments, a performance score may be calculated as:

$$Score = A * wA + T * wT + P * wP + D * wD + S * wS + C * wC - R * wR$$

where, A is path accuracy, T is timing accuracy, P is pressure consistency, D is depth consistency, S is motion smoothness, C is completion, R is risk penalty, and wA through wR are weighting coefficients. Skill level data may be determined by comparing the score and individual component metrics to benchmark thresholds.

Further, credential data may include a user identifier, training module identifier, benchmark identifier, score, component metrics, date, device identifier, evaluator identifier, and cryptographic signature or verification code.

In some embodiments, the present disclosure describes a tattooing system. Further, the tattooing system includes a handheld tattoo device. Further, the handheld tattoo device includes a motor-driven reciprocating needle assembly. Further, the handheld tattoo device includes a stroke-adjustment mechanism (with an actuator) coupled to the needle assembly and able to vary stroke length during operation. Further, the tattooing system includes two or more sensors configured to detect one or more of resistance, torque, vibration, displacement, and motion (related to interaction between the needle and skin). Further, the tattooing system includes a control system coupled to the sensors and stroke-adjustment mechanism. Further, the control system receives sensor data during needle movement. Further, the control system detects changes in skin interaction. Further, the control system generates a control signal based on those changes. Further, the control system adjusts stroke length, frequency, torque, or depth in real time. Further, all actions associated with the control system occur continuously during needle insertion. Further, the control system operates in a closed-loop system. Further, the control system maintains consistent needle penetration across different skin conditions.

In some embodiments, the present disclosure describes a tattoo guidance system. Further, the tattoo guidance system includes a wearable AR device, including a display, an imaging sensor, and a spatial mapping sensor. Further, the tattoo guidance system includes system modules such as a surface mapping module configured for building a dynamic skin model (curvature+deformation), a stencil generation module configured for creating a digital stencil, and a synchronization module configured for aligning the stencil to the skin model and continuously updating position, scale, and orientation. Further, the stencil stays aligned to the skin within a defined tolerance even during movement.

In some embodiments, the present disclosure describes a tattoo execution guidance system. Further, the tattoo execution guidance system includes a vision module configured for tracking a real-time needle position, a reference module configured for storing a digital tattoo path, a comparison module configured for measuring a deviation, and a feedback module configured for generating correction signals. Further, corrections happen in real time during tattooing to reduce deviation.

In some embodiments, the present disclosure describes a non-transitory computer-readable medium storing a tattoo execution file including motion trajectory data (paths), timing data, interaction data (force, resistance, skin contact), machine parameters (stroke, depth, frequency), and adaptive correction rules. Further, the file may run on a tattoo machine. Further, the file reproduces the tattoo process with real-time adjustments.

In some embodiments, the present disclosure describes an integrated tattoo system including a tattoo device (needle+stroke control), an AR wearable device (aligned stencil), a sensor system (detects skin+movement), a control system linking everything. Further, the control system functions include syncing machine with stencil alignment, adjusting tattooing parameters, and updating stencil based on skin movement. Further, as a result machine operation depends on stencil alignment and stencil updates continuously from machine feedback.

Further, in some embodiments, the processor ensures—functionality of various actuator types (micro-linear, piezo), predictive adjustments, <10 ms response time, no interruption during operation, sensor fusion, micro-adjustments, and motion-based adjustments.

In some embodiments, the handheld tattoo device includes an adaptive control system configured to adjust one or more operating parameters during active tattooing based on sensor data representing interaction conditions between the device and a skin surface. The adaptive control system may include micro-linear actuators, piezoelectric actuators, or other electronically controlled actuators configured to perform fine stroke, depth, speed, voltage, torque, frequency, or give adjustments without interrupting operation of the motor. The processor may fuse sensor data from multiple sensors, predict changes in resistance, vibration, displacement, pressure, motion, or other interaction conditions, and generate real-time control outputs, including micro-adjustments and motion-based adjustments, with a response time of less than about 10 milliseconds.

In some embodiments, the system includes wearable augmented reality glasses configured to display and maintain a digital tattoo stencil in alignment with a target skin surface. The augmented reality system may perform three-dimensional skin mapping, anatomical anchoring, predictive alignment, and compensation for skin stretching, compression, deformation, subject movement, or tattoo-device movement. The system may update stencil registration at a rate of at least 30 updates per second and may maintain alignment even when the skin surface or stencil region is partially occluded.

In some embodiments, the system monitors tattoo execution relative to a digital stencil, intended path, or reference tattooing data and provides real-time feedback to an operator. The feedback may include visual cues, augmented reality overlays, haptic alerts, confidence scores, deviation indicators, saturation warnings, or overwork-risk notifications. In response to detected execution deviation, unsafe conditions, overwork, or ink saturation changes, the system may modify one or more operating parameters of the handheld tattoo device.

In some embodiments, the system generates, stores, receives, or executes a tattoo skill file including tattoo execution data, motion trajectories, timing data, ink parameters, saturation data, needle settings, operating parameters, skin-type metadata, anatomical-location metadata, and adaptive correction rules. The tattoo skill file may be platform independent and executable across different tattoo devices, augmented reality systems, robotic systems, semi-autonomous systems, or training platforms through a translation layer that converts the file into device-specific instructions.

In some embodiments, the tattooing system operates as an integrated closed-loop platform in which the handheld tattoo device, sensors, processor, control module, augmented reality glasses, and digital stencil system exchange data bidirectionally during tattooing. The system may use predictive motion adjustment, sensor-based feedback, augmented reality alignment data, and machine-control data to maintain tattoo execution within a predetermined tolerance, including sub-millimeter accuracy, while dynamically coordinating stencil alignment, visual guidance, and operating-parameter adjustment.

In contrast to conventional systems that require manual adjustment of tattoo machine settings and rely on separately applied physical stencils, the disclosed tattooing system provides an integrated real-time adaptive tattoo platform in which a handheld tattoo device, sensor-based needle control, augmented reality guidance, and motion-responsive technique recognition operate together. In some embodiments, the system dynamically adjusts stroke length or other operating parameters during active operation of the tattooing device, without stopping the motor, based on sensor data representing resistance, vibration, displacement, pressure, motion, skin interaction, or ink response. Unlike existing tattoo machines, the system may coordinate machine control with augmented reality stencil placement and alignment, such that visual guidance, skin mapping, stencil tracking, and tattoo device operation are updated together during tattooing. The system may further recognize artist motion, hand speed, dwell time, acceleration, directional change, or movement pattern to infer a tattooing technique, such as lining, shading, color packing, or stippling, and automatically adapt machine operation to the inferred technique.

In some embodiments, the tattooing system may operate in a plurality of modes of operation. In a standalone machine mode, the handheld tattoo device may operate independently using onboard sensors, a processor, and a control module to adjust one or more operating parameters during tattooing. In a partial system mode, the handheld tattoo device may communicate with one or more external devices, sensors, displays, processors, or user interfaces to provide selected functions such as guidance, monitoring, feedback, data storage, or parameter adjustment. In a full augmented reality and artificial intelligence system mode, the handheld tattoo device, wearable augmented reality glasses, sensor systems, artificial intelligence modules, and control modules may operate together to provide real-time stencil alignment, machine control, motion or technique recognition, execution feedback, and adaptive tattooing guidance.

In one exemplary use case, during tattooing, the system detects an increase in resistance between the handheld tattoo device and the skin surface based on sensor data from one or more sensors. In response, the control module automatically adjusts one or more operating parameters, such as stroke length, needle depth, motor speed, torque, or operating frequency, while the operation of the motor continues. At the same time, the augmented reality system maintains alignment of the digital stencil with the skin surface, thereby allowing the tattooing operation to continue with improved consistency, guidance, and accuracy.

In some embodiments, the disclosed tattooing system improves technical coordination between a handheld tattoo machine and a wearable augmented reality guidance device by integrating adaptive machine control, spatial stencil registration, machine-path tracking, and real-time feedback into a single closed-loop operating environment. The handheld tattoo device may include a housing, grip, motor, drive shaft, cam or equivalent motion-conversion assembly, needle bar interface, cartridge interface, and reciprocating needle assembly, while the wearable augmented reality glasses may include one or more displays, cameras, depth sensors, inertial sensors, processors, and communication interfaces. During tattoo execution, sensor data from the tattoo machine and spatial data from the glasses may be processed together so that the system can correlate needle movement, skin interaction, stencil position, and operator motion rather than treating stencil placement and machine operation as isolated tasks. This arrangement addresses implementation challenges associated with maintaining a tattoo path on a deformable anatomical surface while a motor-driven needle assembly is actively interacting with skin, because the same operating architecture can update machine parameters and guidance overlays in response to sensed resistance, displacement, position, movement, and surface deformation. As a result, the disclosed system can maintain closer correspondence between the intended digital stencil and the actual tattoo execution path while also adjusting the mechanical behavior of the tattoo machine during the procedure.

In some embodiments, the adaptive tattoo machine improves penetration consistency by using a dynamically controllable stroke-adjustment mechanism that changes stroke length during active operation without requiring interruption of motor function. The stroke-adjustment mechanism may be implemented using a linear actuator, solenoid, piezoelectric device, servo mechanism, magnetic positioning assembly, cam translation system, or equivalent adjustment structure coupled to the needle drive mechanism. A control processor may receive displacement, resistance, torque, vibration, pressure, skin-contact, or needle-excursion signals and determine whether the current stroke setting should be increased, decreased, or micro-adjusted to maintain a target interaction profile with the target skin surface. For example, when a local area of skin presents increased resistance or altered elasticity, the processor may adjust stroke length, motor speed, voltage, torque, give, frequency, or penetration depth while the motor continues to drive the reciprocating needle assembly. The technical effect is that stroke configuration becomes a real-time controlled parameter rather than a static preselected setting, enabling the tattoo machine to respond to changing skin conditions, anatomical contours, and operator motion.

In some embodiments, the disclosed sensor-assisted control architecture improves control accuracy by fusing multiple categories of machine and skin-interaction data before producing control outputs. The sensor system may detect resistance, torque, vibration, displacement, acceleration, orientation, pressure, skin contact, needle excursion, ink deposition, optical characteristics, spectral characteristics, cartridge state, and environmental conditions, and the control processor may combine these signals to determine real-time operating conditions and skin response. By analyzing multiple sensor channels, the control processor may distinguish between different causes of machine behavior, such as increased resistance caused by skin elasticity, a displacement anomaly caused by needle motion, a vibration signature caused by cartridge behavior, or an optical response indicating altered pigment density. This fused sensor configuration reduces reliance on any single sensor measurement and enables the control algorithm to generate more technically appropriate adjustments, such as reducing aggressiveness when vibration and pressure exceed a threshold, increasing deposition when optical feedback indicates insufficient saturation, or issuing a warning when contact conditions suggest depth risk. The resulting system operation is more stable because the machine-control decision is based on a richer state representation of the tattooing interaction.

In some embodiments, the closed-loop control algorithm improves the responsiveness of the tattoo machine by continuously receiving sensor data, determining deviations or changing operating conditions, and applying parameter adjustments during the tattooing sequence. The processor may implement rule-based logic, predictive algorithms, or machine learning models trained on prior tattooing data to evaluate whether sensed changes indicate impending resistance variation, unsafe depth, inadequate ink deposition, over-saturation, under-saturation, or a technique transition. In one implementation, the control loop may receive a stream of displacement and resistance readings, compare the readings with operating thresholds or learned skin-response profiles, and output adjustment commands to a stroke actuator, motor driver, voltage controller, torque controller, give controller, or frequency controller. In another implementation, the system may apply parameter corrections with low latency after receiving updated sensor data, thereby reducing the delay between a detected change at the needle-skin interface and a corresponding machine response. This control-loop arrangement improves the technical operation of the tattoo machine by enabling the device to maintain target operating behavior across varying skin surfaces, hand speeds, and contact conditions.

In some embodiments, the augmented reality glass-based stencil system improves stencil placement and maintenance by replacing a static physical transfer with a spatially registered digital stencil that can be generated, displayed, manipulated, and re-aligned relative to the target skin surface. Further, the glass may use cameras, depth sensors, inertial sensors, and processors to map the target surface, detect anatomical features, identify skin curvature, and display a stencil through a head-mounted or wearable display. The digital stencil may be translated, rotated, scaled, mirrored, warped, curved, opacity-adjusted, layered, segmented, recalled, saved, duplicated, or otherwise manipulated using gesture input, voice input, touch input, physical controls, remote input, or combinations of such interfaces. Because the stencil is maintained as a digital object associated with spatial mapping data, the system can preserve placement data, recall prior configurations, adjust opacity during different execution stages, and adapt stencil geometry to body curvature. The technical effect is improved alignment control, repeatability, and operator visibility during tattoo planning and execution, particularly when the target surface is curved, moving, or partially obstructed by the tattoo machine or operator hand.

In some embodiments, the anchoring and tracking framework improves stencil registration by locking a digital stencil to detected anatomical landmarks, body contours, reference markers, fiducials, or mapped skin topography rather than relying solely on a fixed display coordinate. The spatial tracking system may detect breathing-induced movement, posture changes, localized skin stretch, compression, subject movement, or deformation caused by needle contact, and the synchronization module may update stencil position, scale, curvature, and orientation in response to the detected surface changes. For example, when skin is stretched by the operator during line work, the system may update the digital stencil so that the displayed reference remains conformal to the deformed surface rather than drifting relative to the actual skin. In another operating scenario, when a subject changes posture or breathes during tattooing, the alignment module may maintain the stencil within a predetermined tolerance relative to the target area. This anchoring arrangement improves technical reliability of augmented reality guidance by maintaining correspondence between digital planning data and a moving physical surface.

In some embodiments, the real-time guidance system improves execution accuracy by comparing actual needle position, actual machine path, or actual ink placement with a reference stencil or intended tattoo path and generating correction cues based on detected deviation. A computer vision module may capture real-time imagery of the target skin surface, while a comparison module may determine positional error, path offset, missed areas, excess dwell, overwork risk, or depth-related risk. The correction module may generate visual overlays through the glasses, auditory alerts, haptic feedback through the machine or wearable device, directional arrows indicating a corrected stroke path, depth indicators, warning regions, or suggested trajectory changes. When the system determines that the needle path is diverging from the reference stencil, the overlay may indicate the direction and magnitude of correction while the control processor optionally modifies machine parameters to reduce damage risk or improve line consistency. This feedback architecture improves the technical execution workflow by converting live visual and motion data into actionable machine-side and user-side guidance signals.

In some embodiments, the motion-responsive technique adaptive control layer improves parameter selection by classifying or inferring tattooing technique from hand movement of the tattoo machine during active operation. One or more accelerometers, gyroscopes, inertial measurement units, optical flow sensors, or related motion sensors may detect movement speed, acceleration, directional change, dwell time, tapping patterns, and sweeping motion, and a processor may analyze those signals to infer whether the operator is performing lining, shading, color packing, or stippling. Slower deliberate movement may correspond to lining and may cause the control module to increase stroke length or penetration consistency; sweeping motion may correspond to shading and may cause the control module to reduce stroke aggressiveness; slower high-contact movement may correspond to color packing and may cause saturation-oriented settings to be selected; and intermittent tapping movement may correspond to stippling and may cause the machine to shift to stippling settings. By combining hand speed data with skin resistance data before adjusting stroke length, needle frequency, motor speed, torque, give, penetration depth, or ink delivery characteristics, the system improves technique-specific control without requiring the operator to stop the machine and manually reconfigure settings.

In some embodiments, the disclosed system improves adaptive control reliability by generating a confidence score corresponding to an inferred tattooing technique before changing one or more tattooing parameters. The processor may evaluate movement features such as hand speed, acceleration, dwell time, directional change, and periodicity together with skin-interaction features such as resistance, contact pressure, and vibration to determine whether the detected motion pattern sufficiently matches a technique profile. When confidence exceeds a configurable threshold, the control module may transition to a corresponding lining, shading, color packing, or stippling operating mode; when confidence remains below the threshold, the system may maintain the current operating state, generate a user prompt, or apply only limited parameter adjustment. This confidence-mediated transition reduces abrupt or erroneous control changes during ambiguous movements, such as when an artist transitions between a line and a shaded region or temporarily pauses for repositioning. The technical effect is smoother mode switching, reduced parameter oscillation, and greater consistency during mixed-technique tattoo sequences.

In some embodiments, the ink absorption and skin response adaptation system improves pigment deposition control by using optical or spectral sensors to analyze ink deposition into the skin surface and by applying machine learning models that predict absorption characteristics. The system may detect pigment density, over-saturation, under-saturation, skin type, hydration, elasticity, or related skin-response variables, and the control processor may adjust needle depth, stroke speed, stroke length, frequency, torque, give, or ink flow rate based on the detected state. For example, if optical feedback indicates over-saturation, the control system may reduce ink flow or reduce penetration aggressiveness; if the sensor data indicates insufficient saturation, the system may increase deposition-oriented settings while monitoring safety thresholds. The feedback loop may continuously refine the model based on real-time results, allowing the system to adapt to skin variation across different anatomical regions and across different stages of the same tattoo session. This improves technical control of pigment placement by linking optical or spectral evidence of deposition quality to mechanical and electrical control of the tattoo machine.

In some embodiments, the predictive motion compensation system improves alignment stability by forecasting future movement trajectories of the target skin surface and compensating before the movement fully disrupts stencil registration or needle-path accuracy. A motion tracking system may detect subject body movement, breathing-induced movement, tremor, posture changes, or localized displacement, and an artificial intelligence prediction engine may estimate near-term movement based on prior movement data, real-time tracking data, or learned motion patterns. The compensation module may adjust stencil alignment in advance of the movement, modify machine operation, filter tremor components, or generate guidance overlays that account for predicted displacement. For example, when breathing causes periodic movement of a torso region, the system may predict the next phase of movement and adjust the digital stencil position or issue timing guidance to the operator. This produces a technical improvement in dynamic registration by reducing mismatch between the visual reference, the target skin surface, and the actual machine path during voluntary or involuntary movement.

In some embodiments, the tattoo operating system improves orchestration of tattoo execution by providing a centralized control architecture that coordinates the tattoo device, augmented reality glasses, sensor systems, motion data, skin-interaction data, visual data, and artificial intelligence decision logic. The operating system may include a real-time data processing engine, stencil rendering module, motion control module, skin analysis module, artificial intelligence decision engine, synchronization layer, application programming interface, plugin architecture, and sandboxed software execution framework. The synchronization layer may maintain alignment between physical execution and digital representations, while modular software components may independently process stencil display, machine control, skin analysis, training overlays, and execution enhancements. This architecture addresses the implementation challenge of coordinating heterogeneous hardware and software modules operating at different update rates and data formats. The technical effect is a scalable processing platform in which visualization, machine actuation, sensing, model inference, logging, and external application integration can operate in a coordinated real-time workflow.

In some embodiments, the tattoo skill file format improves reproducibility and interoperability by encoding tattoo execution as structured machine-readable data rather than as an unstructured recording or operator memory. The skill file may include motion trajectory data, temporal execution data, force and interaction data, needle configuration data, ink deposition parameters, contextual metadata, and adaptive correction rules. A runtime execution engine may interpret the motion trajectory, timing, sequencing, speed, pressure, resistance, skin-contact parameters, depth, stroke length, frequency, saturation thresholds, layering instructions, skin type, anatomical location, and environmental conditions to generate control signals for a tattoo device, augmented reality system, robotic device, or semi-autonomous execution system. A cross-platform translation layer may convert the platform-independent skill file into machine-specific instructions while accounting for device-specific mechanical tolerances. This improves technical interoperability by allowing recorded or generated tattoo processes to be replayed, evaluated, modified, and executed across heterogeneous tattoo hardware and guidance systems.

In some embodiments, the semi-autonomous and robotic execution framework improves controlled tattoo execution by combining predefined tattoo instructions, real-time sensor feedback, and human override capability. A robotic or motor-assisted tattoo device may receive control instructions from a skill file, artificial intelligence controller, or execution engine, while sensors monitor skin interaction, surface movement, pressure, resistance, and ink deposition. In a semi-autonomous mode, automated line tracing or shading execution may proceed under human supervision, and a human override interface may allow the operator to guide, pause, correct, or intervene in execution. In a more automated mode, a robotic arm or automated positioning system may control tattooing instrument position, motion, pressure, and ink deposition while a safety system detects unsafe conditions and halts operation. This improves execution repeatability and safety by linking automated motion control with skin-aware feedback and by preserving intervention pathways when detected deviation or risk exceeds threshold limits.

In some embodiments, the skin digital twin engine improves planning and execution by generating a three-dimensional model of the subject's skin surface and using simulation to predict tattoo outcomes before or during execution. A 3D scanning module may capture anatomical curvature and topography, while a simulation engine may represent physical or biological properties such as elasticity, hydration, pigmentation, ink spread, color blending, tissue response, or healing-related appearance. A prediction module may simulate ink dispersion, fading over time, scar formation risk, or long-term appearance, and the visualization interface may display predicted healed results or pre-execution tattoo placement through the augmented reality glasses. During tattooing, real-time sensor input may update the digital twin, and simulation output may inform machine parameter adjustment or augmented reality overlay adjustment. This produces a technical improvement by allowing design placement, machine control, and safety guidance to be informed by a dynamic computational representation of the target skin surface rather than by static visual observation alone.

In some embodiments, the disclosed safety monitoring system improves operational control by detecting unsafe tattooing conditions and generating alerts or responsive control actions before the system continues into a higher-risk operating state. One or more sensors may detect excessive resistance, abnormal vibration, excessive pressure, depth risk, overwork risk, unsafe pigmentation response, inflammation, irritation, abnormal skin response, or predicted skin trauma thresholds. A processor may compare the detected condition with safety thresholds and may generate a haptic alert, visual warning through the augmented reality glasses, audible notification, parameter reduction, execution pause, lockout state, or automatic halt. For example, if the system detects a depth condition associated with predicted skin damage risk, the glasses may display a depth warning while the tattoo machine reduces penetration depth, stroke aggressiveness, or motor output. This safety framework improves reliability by connecting sensor-derived risk detection with both operator-facing feedback and machine-side intervention.

In some embodiments, the biometric authentication and lockout framework improves controlled operation of the tattoo machine and integrated ecosystem by requiring identity verification before machine activation, skill file use, cloud access, or communication with the wearable guidance system. The tattoo machine, glasses device, or operating system may authenticate a user biometrically, using the authentication result to permit operation, pair authorized devices, load user-specific settings, or access licensed content. If the system detects unauthorized use, authentication failure, or an unrecognized machine-glasses pairing, the tattoo machine may enter a lockout state, the operating system may restrict access to control functions, or the cloud platform may deny retrieval of protected skill files. This security behavior reduces unauthorized execution of machine settings, technique files, or automated tattoo sequences and improves technical traceability by associating operational data, user profiles, and execution logs with authenticated operators.

In some embodiments, encrypted communication between system components improves data protection and command integrity across the tattoo machine, wearable augmented reality glasses, external devices, cloud infrastructure, remote expert terminals, and operating system modules. The communication framework may transmit control instructions, sensor streams, visual data, stencil registration data, skill files, firmware updates, user profiles, model updates, and session logs using encrypted wireless or wired links. When the glasses generate control instructions for the tattoo machine based on visual analysis, the encrypted connection may protect those instructions from unauthorized modification while maintaining a latency target suitable for active guidance and control. Similarly, encrypted skill files may prevent unauthorized copying or execution of technique data distributed through a cloud repository or marketplace. This security architecture improves trusted operation of distributed tattoo workflows in which safety-critical control commands and proprietary execution data are exchanged between multiple computing nodes.

In some embodiments, the integrated illumination system improves visual sensing and operator visibility by positioning illumination adjacent to or near the cartridge, needle region, or active working zone of the tattoo machine. The illumination output may be controlled based on ambient conditions, detected tattooing conditions, visual sensor requirements, or operator settings, and may enhance visibility of the needle, cartridge, stencil, ink deposition, and local skin response. For example, when the glasses or machine-mounted cameras require clearer imagery for computer vision comparison of actual ink placement against the reference stencil, the illumination system may increase brightness or adjust output near the working zone. When ambient lighting is sufficient or reflective glare interferes with visual detection, the system may reduce illumination or modify output intensity. This improves both human-machine interface operation and computer vision reliability by stabilizing visual conditions at the needle-skin interaction region.

In some embodiments, the remote collaboration system improves distributed guidance by allowing a remote computing system or expert terminal to provide real-time assistance, annotations, overlays, or instructions to a local user through the wearable augmented reality display. The communication system may transmit visual imagery, stencil state, machine state, sensor data, session context, and execution metrics to the remote system, and the remote expert may return guidance overlays, marked correction regions, suggested path changes, or instructional cues. The local glasses may present these annotations in spatial registration with the target skin surface, while the tattoo operating system may coordinate the remote overlay with current stencil alignment, machine path, and skin-tracking state. This arrangement improves technical collaboration by converting remote input into spatially anchored augmented reality guidance rather than requiring the local operator to mentally translate verbal instructions into machine movement.

In some embodiments, the training and certification framework improves objective evaluation of tattoo technique by capturing execution data and comparing user actions with benchmarks, expert patterns, or recorded skill files. The wearable glasses may display guided execution overlays, simulated practice overlays, real-time corrective cues, shading gradients, directional arrows, and expert tattoo patterns, while the performance tracking system monitors accuracy, timing, motion trajectory, hand speed, dwell time, technique classification, depth risk, and deviation from an intended path. An evaluation engine may generate scores based on accuracy, timing, technique, or combinations thereof, and a certification module may produce credentials corresponding to performance tiers. Historical performance data may be used to personalize training by identifying recurring deviation types, unstable movement patterns, insufficient saturation events, or timing inconsistencies. This improves training consistency by converting live tattoo execution into measurable data streams and feedback events that can be replayed, scored, and progressively refined.

In some embodiments, the cloud-based tattoo model repository and distributed operating system network improve scalability by storing and synchronizing tattoo sessions, skill files, user profiles, performance analytics, model updates, training data, and compatible execution content across devices. A cloud infrastructure may distribute updated machine learning models, synchronized stencil configurations, recorded sessions, replayable augmented reality procedures, firmware updates, and licensed skill files to authorized tattoo operating systems. An anonymized data aggregation layer may update shared intelligence models based on tattoo session data while preserving user and session control through authentication and encryption. The technical effect is that improvements learned from one operating environment, such as updated technique classification parameters, improved skin-response models, or refined motion compensation rules, may propagate to other compatible devices through controlled synchronization rather than requiring manual device-by-device reconfiguration.

In some embodiments, the application programming interface and plugin architecture improve extensibility of the tattoo operating system by allowing third-party or separately installed modules to interact with core functions such as stencil rendering, motion control, skin analysis, artificial intelligence decision logic, training systems, and execution enhancements. Software modules may operate in a sandboxed architecture so that an installed design tool, training extension, analytics dashboard, monetized software extension, or execution enhancement can access permitted data or control interfaces without interfering with safety-critical control loops. Access may be controlled using authentication credentials, and firmware or software updates may be deployed over the air to maintain compatibility with the machine, glasses, sensors, and cloud services. This modular architecture improves platform maintainability because new visualization features, model updates, analytics functions, or skill-file tools can be added without restructuring the entire real-time control framework.

In some embodiments, execution logs and replayable session records improve traceability by recording machine parameters, sensor readings, stencil state, operator motion, visual guidance events, safety alerts, control adjustments, and resulting tattoo execution data during a procedure. The tattoo operating system may store the logs locally, transmit them to an external device, or synchronize them with a cloud repository for later playback, training, analysis, certification, debugging, or model improvement. In a replay mode, the glasses may display the recorded stencil, machine path, correction cues, and timing data in augmented reality, allowing an operator or evaluator to inspect how the tattoo sequence progressed relative to the intended design. This record-keeping framework improves auditability and technical diagnostics by preserving the relationship between sensed conditions, control decisions, user actions, and visible execution outcomes.

In some embodiments, the medical-grade skin analysis layer improves parameter selection by using sensors and analysis engines to detect biological skin characteristics before or during tattooing. One or more sensors may detect skin thickness, hydration, vascularity, pigmentation, melanin density, vascular mapping data, inflammation, irritation, abnormal response, or prior skin condition information obtained through a clinical integration interface. The analysis engine may classify skin condition and calculate safe depth ranges, while a safety module may determine tattooing parameters that reduce excessive skin trauma risk. If the system predicts adverse outcomes or detects threshold risk, it may provide recommendations to the operator, adjust stroke length, reduce penetration depth, alter ink deposition settings, or halt machine operation. This improves safety-aware control because machine parameters are derived from measured or integrated skin-specific data rather than from a fixed operating configuration.

In some embodiments, the adaptive correction rules within a tattoo skill file improve execution robustness by allowing a recorded or generated tattoo process to change in response to real-time feedback rather than following a rigid path. The skill file may define nominal trajectories, timing, force, pressure, resistance, needle configuration, ink deposition settings, layering instructions, and saturation thresholds, while also specifying correction rules that respond to deviation, skin movement, over-saturation, under-saturation, or safety thresholds. During playback, the runtime execution engine may compare real-time execution against the encoded instructions, generate deviation scores, pause execution if risk limits are exceeded, or adjust control signals for the tattoo device or robotic system. This produces a technical improvement in reproducible tattoo execution because the encoded technique can accommodate variation in skin condition, machine tolerance, anatomical location, and operator intervention while preserving the intended procedural structure.

In some embodiments, the compatibility engine and marketplace infrastructure improve distribution of digital tattoo techniques by treating tattoo skill files as executable digital assets that can be accessed, purchased, licensed, rated, analyzed, and replayed across compatible devices. The cloud platform may store encrypted skill files, associate usage data with licensing or monetization records, distribute creator royalties, and expose performance analytics indicating how a file executes across different machines, anatomical areas, skin types, or operator skill levels. A compatibility engine may determine whether a particular tattoo device, robotic system, glasses configuration, or operating system module can interpret the file and may apply translation rules for hardware tolerances, stroke ranges, sensor availability, or execution modes. This improves digital interoperability and controlled distribution by coupling access management, device compatibility, and execution analytics in a unified cloud-connected ecosystem.

In some embodiments, the hybrid human and artificial intelligence collaboration system improves control allocation by allowing authority over tattoo execution to shift between a human artist and an assistance engine based on deviation, confidence, safety, or technique state. A tattoo device operated by the human artist may be monitored by computer vision, motion tracking, skin-response sensing, and comparison modules that determine whether the current execution remains within acceptable limits relative to the stencil, skill file, or predicted skin response. If deviation exceeds a threshold, the artificial intelligence system may provide intensified correction cues, temporarily assume partial control of machine parameters, reduce aggressiveness, stabilize output, or guide the operator back to the intended path. If execution returns within tolerance, control may shift back toward ordinary human operation. This dynamic control-sharing arrangement improves robustness by allowing the system to intervene proportionally to detected execution risk rather than operating only in fully manual or fully automated modes.

In some embodiments, the real-time firmware update and model update framework improves lifecycle adaptability of the tattoo machine, glasses, and operating system by distributing updated control logic, sensor-processing routines, skin-response models, technique classifiers, stencil-rendering modules, or safety thresholds through authenticated update channels. The operating system may validate update compatibility with installed hardware, preserve prior operating profiles, and apply updates to individual modules without disrupting unrelated functions. For example, an updated hand-speed classification model may be installed while maintaining existing user profiles and skill-file compatibility, or a refined ink absorption model may be deployed to improve optical feedback interpretation. This improves long-term technical maintainability because the adaptive control and guidance behavior can evolve through controlled software and firmware updates while retaining interoperability with the broader tattoo ecosystem.

In some embodiments, personalized user profiles improve adaptive control by storing artist-specific movement profiles, preferred settings, prior technique data, training history, stencil manipulation preferences, authentication credentials, and session-specific calibration parameters. The processor may use these profiles to interpret motion patterns more accurately, because the same absolute hand speed or dwell time may correspond to different technique intent for different users. For example, an artist-specific movement profile may help distinguish a slow lining pass from a color packing movement by combining historical motion features with current resistance and skin-contact data. The system may also recall preferred stencil opacity, saved placement configurations, or machine behavior settings for similar anatomical locations or design types. This improves technical personalization by allowing the machine-control and augmented reality guidance frameworks to adapt to repeatable user-specific behavior while remaining governed by safety and feedback constraints.

In some embodiments, analytics dashboards improve operational monitoring by presenting processed session data, performance metrics, safety events, deviation scores, saturation measures, technique classifications, machine adjustments, and training trends in a structured interface. The dashboard may receive data from local logs, cloud-synchronized records, skill file playback, certification modules, or distributed operating system nodes. During post-session review, the dashboard may identify repeated overwork warnings, frequent stencil deviation zones, unstable hand-speed transitions, insufficient saturation regions, or recurring safety threshold events. During fleet or studio management, aggregated dashboard data may support device maintenance, user training, model improvement, usage tracking, licensing, or quality review. This data-processing layer improves technical observability by transforming raw sensor and execution records into actionable metrics tied to machine operation and tattoo workflow outcomes.

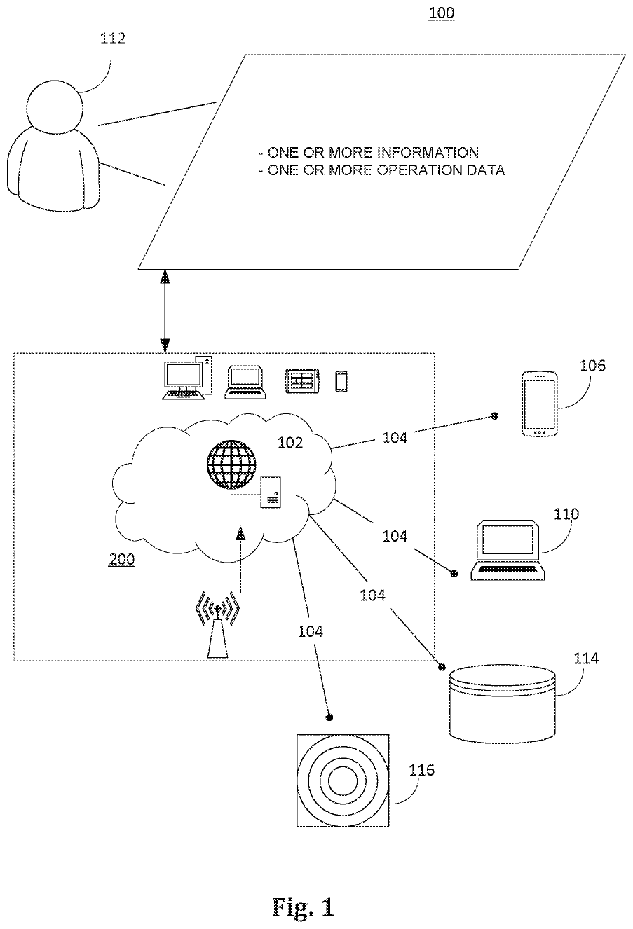

FIG. 1 is an illustration of an online platform 100 consistent with various embodiments of the present disclosure. By way of non-limiting example, the online platform 100 may be hosted on a centralized server 102, such as, for example, a cloud computing service. The centralized server 102 may communicate with other network entities, such as, for example, a mobile device 106 (such as a smartphone, a laptop, a tablet computer etc.), other electronic devices 110 (such as desktop computers, server computers etc.), databases 114, and sensors 116 over a communication network 104, such as, but not limited to, the Internet. Further, users of the online platform 100 may include relevant parties such as, but not limited to, users, administrators, service providers, service consumers, and so on. Accordingly, in some instances, electronic devices operated by the one or more relevant parties may be in communication with the platform.

A user 112, such as the one or more relevant parties, may access online platform 100 through a web-based software application or browser. The web-based software application may be embodied as, for example, but not limited to, a website, a web application, a desktop application, and a mobile application compatible with a computing device 200.

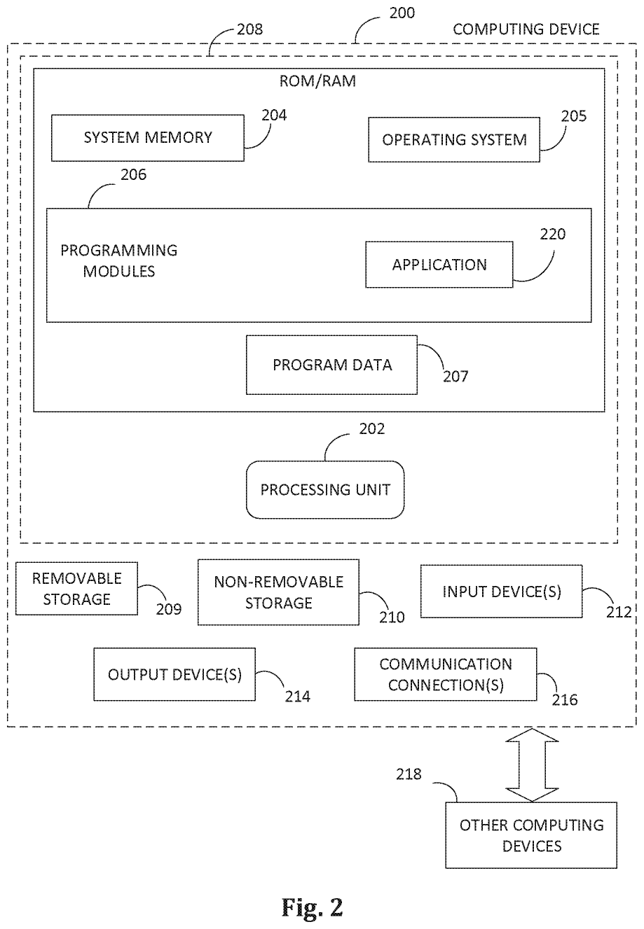

With reference to FIG. 2, a system consistent with an embodiment of the disclosure may include a computing device or a cloud service, such as a computing device 200. In a basic configuration, the computing device 200 may include at least one processing unit 202 and a system memory 204. Further, the at least one processing unit 202 may include a processor. Moreover, the processor may be and/or may include general-purpose processors, specialized processors, machine learning accelerators, multi-core processors, vector processors, digital signal processors, tensor accelerators, neural accelerators, graphics engines, or various kinds of specialized integrated circuits including FPGAs, ASICs, ASSPs, SoCs, and CPLDs. Depending on the configuration and type of the computing device 200, the system memory 204 may comprise, but is not limited to, a volatile memory (e.g., random-access memory (RAM)), non-volatile memory (e.g., read-only memory (ROM)), a flash memory, or any combination. System memory 204 may include an operating system 205, one or more programming modules 206, and a program data 207. Operating system 205, for example, may be suitable for controlling computing device 200's operation. In one embodiment, the one or more programming modules 206 may include image-processing modules, machine learning modules, etc. Furthermore, embodiments of the disclosure may be practiced in conjunction with a graphics library, other operating systems, or any other application program, and are not limited to any particular application or system. This basic configuration is illustrated in FIG. 2 by those components within a dashed line 208.

Computing device 200 may have additional features or functionality. For example, the computing device 200 may also include additional data storage devices (removable and/or non-removable) such as, for example, magnetic disks, optical disks, or tape. Such additional storage is illustrated in FIG. 2 by a removable storage 209 and a non-removable storage 210. Computer storage media may include volatile and non-volatile, removable and non-removable media implemented in any method or technology for storage of information, such as computer-readable instructions, data structures, program modules, or other data. System memory 204, removable storage 209, and non-removable storage 210 are all computer storage media examples (i.e., memory storage). Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information and which can be accessed by computing device 200. Any such computer storage media may be part of the computing device 200. Computing device 200 may also have input device(s) 212, such as a keyboard, a mouse, a pen, a sound input device, a touch input device, a location sensor, a camera, a biometric sensor, etc. Output device(s) 214, such as a display, speakers, a printer, etc., may also be included. Further, the input device(s) 212 and the output device(s) 214 may be suitable for multimodal interaction with a user. The aforementioned devices are examples, and others may be used.

Computing device 200 may also contain a communication connection 216 that may allow device 200 to communicate with other computing devices 218, such as over a network in a distributed computing environment, for example, an intranet or the Internet. Further, the communication connection may be and/or may include a communication interface, a network interface, etc. Communication connection 216 is one example of communication media. Communication media may typically be embodied by computer-readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer-readable media as used herein, may include both storage media and communication media.

As stated above, a number of program modules and data files may be stored in the system memory 204, including the operating system 205. While executing on the at least one processing unit 202, the one or more programming modules 206 (e.g., application 220 such as a media player) may perform processes including, for example, one or more stages of methods, algorithms, systems, applications, servers, and databases as described above. The aforementioned process is an example, and the at least one processing unit 202 may perform other processes. Other programming modules that may be used in accordance with embodiments of the present disclosure may include machine learning applications.

Generally, consistent with embodiments of the disclosure, program modules may include routines, programs, components, data structures, and other types of structures that may perform particular tasks or that may implement particular abstract data types. Moreover, embodiments of the disclosure may be practiced with other computing device and/or computer system configurations, including hand-held devices, general-purpose graphics processor-based systems, multiprocessor systems, microprocessor-based or programmable consumer electronics, application-specific integrated circuit-based electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations, such as, for example, AND, OR, and NOT, including but not limited to mechanical, optical, fluidic, and quantum technologies. In addition, embodiments of the disclosure may be practiced within a general-purpose computer or in any other circuits or systems.

Embodiments of the disclosure, for example, may be implemented as a computer process (method), a computing system, or as an article of manufacture, such as a computer program product or computer-readable media. The computer program product may be a computer storage medium readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device.

The computer-usable or computer-readable medium may be, for example, but not limited to, an electronic, magnetic, optical, electromagnetic, infrared, or semiconductor system, apparatus, device, or propagation medium. More specific computer-readable medium examples (a non-exhaustive list), the computer-readable medium may include the following: an electrical connection having one or more wires, a portable computer diskette, a random-access memory (RAM), a read-only memory (ROM), an erasable programmable read-only memory (EPROM or Flash memory), an optical fiber, and a portable compact disc read-only memory (CD-ROM). Further, the computer-readable medium may be and/or may include one or more non-transitory computer readable media. Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Embodiments of the present disclosure, for example, are described above with reference to block diagrams and/or operational illustrations of methods, systems, and computer program products according to embodiments of the disclosure. The functions/acts noted in the blocks may occur out of the order as shown in any flowchart. For example, two blocks shown in succession may in fact be executed substantially concurrently, or the blocks may sometimes be executed in the reverse order, depending upon the functionality/acts involved.

While certain embodiments of the disclosure have been described, other embodiments may exist. Furthermore, although embodiments of the present disclosure have been described as being associated with data stored in memory and other storage mediums, data can also be stored on or read from other types of computer-readable media, such as secondary storage devices, like hard disks, solid state storage (e.g., USB drive), or a CD-ROM, a carrier wave from the Internet, or other forms of RAM or ROM. Further, the disclosed methods' stages may be modified in any manner, including by reordering stages and/or inserting or deleting stages, without departing from the disclosure.

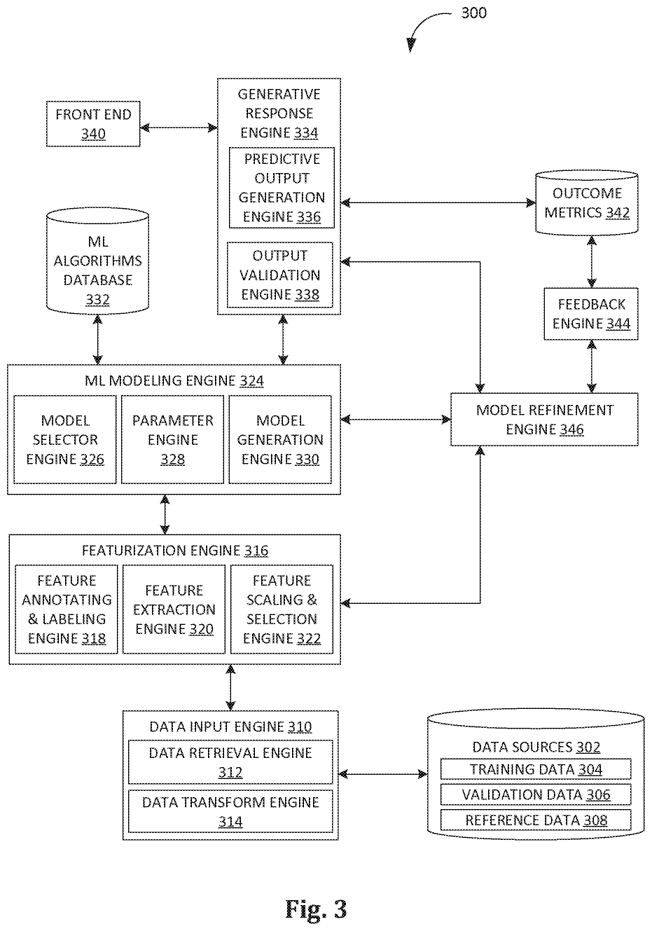

FIG. 3 is a block diagram illustrating a machine-learning system 300 for implementing various embodiments of this disclosure, in accordance with some embodiments. Although the disclosed machine-learning system 300 depicts particular system components and an arrangement of such components, the given depiction is to facilitate a discussion of the present technology and should not be considered limiting unless specified in the appended claims. For example, some components that are illustrated as separate, may be combined with other components and some components may be divided into separate components.

Accordingly, the machine-learning system 300 may include a plurality of interrelated modules and engines configured to implement a machine-learning pipeline. Further, the machine-learning system 300 may include a data sources module 302 that is made up of a training data repository 304, a validation data repository 306, and a reference data repository 308, each repository being configured to store respective classes of input records and reference information. Further, the machine-learning system 300 may include a data input engine 310 configured to receive data from the data sources module 302. Further, the data input engine 310 may include a data retrieval engine 312 configured to access and ingest data from the repositories (304, 306, 308), and a data transform engine 314 configured to perform initial normalization, parsing and format conversion on the ingested data. Further, the data input engine 310 may be implemented on a computing device.

Further, the machine-learning system 300 may include a featurization engine 316 configured to prepare temporal and predictive representations of transformed data. Further, the featurization engine 316 may include a feature annotating & labeling engine 318 for applying labels and annotations to data instances, a feature extraction engine 320 for deriving feature vectors and candidate predictors, and a feature scaling & selection engine 322 for performing numerical scaling, dimensionality reduction and selection of salient features. Further, the featurization engine 316 may be implemented on the computing device.

Further, the machine-learning system 300 may include a machine learning (ML) modeling engine 324 configured to construct predictive models from selected features. Further, the ML modeling engine 324 may include a model selector engine 326 for selecting among candidate model classes, a parameter engine 328 for determining and tuning hyper parameters, and a model generation engine 330 for instantiating and training model artifacts according to selected architectures and parameters. Further, the machine-learning system 300 may include an ML algorithms database 332 configured to store algorithmic implementations, model templates and associated metadata and to be accessible by components of the ML modeling engine 324. Further, the ML modeling engine 324 may be implemented on the computing device.

Further, the machine-learning system 300 may include a generative response engine 334 configured to produce user-facing outputs based on the trained models. Further, the generative response engine 334 may include a predictive output generation engine 336 for generating predictions or synthesized responses and an output validation engine 338 for verifying, filtering and validating generated outputs against predefined criteria and reference data. Further, the machine-learning system 300 may include a front end 340 configured to present validated outputs to end users and to collect interaction signals. Further, the machine-learning system 300 may include an outcome metrics module 342 configured to compute performance measures, accuracy statistics and other evaluation metrics derived from model outputs and user interactions. Further, the generative response engine 334 may be implemented on the computing device.

Further, the machine-learning system 300 may include a feedback engine 344 configured to aggregate outcome metrics and user feedback and to format such information for reuse. Further, the machine-learning system 300 may include a model refinement engine 346 configured to receive feedback from the feedback engine 344 and the outcome metrics module 342, and to effect iterative updates to the ML modeling engine 324 and to the ML algorithms database 332. Further, the components are communicatively coupled so that data and control signals are exchanged among the repositories (304, 306, 308), the data input engine 310, the featurization engine 316, the ML modeling engine 324 (with algorithmic support from the ML algorithms database 332), the generative response engine 334 and the front end 340 for output generation. Further, the outcome metrics 342 and the feedback engine 344 provide closed-loop signals to the model refinement engine 346 to enable retraining, parameter adjustment and algorithm selection, thereby enabling cooperative execution of data acquisition, feature engineering, model construction, output generation, validation, evaluation and iterative refinement within the disclosed machine-learning system 300. Further, the feedback engine 344 may be implemented on the computing device.

Any or each engine of the machine-learning system 300 may be and/or may include a module (e.g., a program module), which may be a hardware unit configured to be used with other components or a part of a program that performs a particular function. Further, any or each engine of the machine-learning system 300 may be implemented using a computing device.

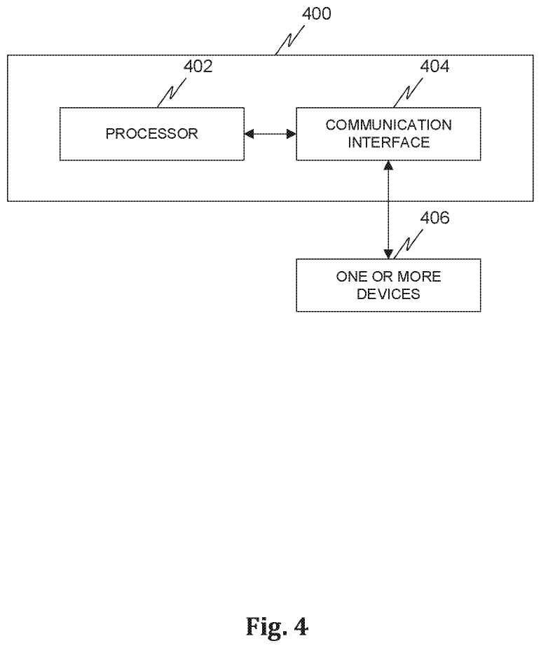

FIG. 4 illustrates a block diagram of a system 400 facilitating the management and creation of tattoos, in accordance with some embodiments. Accordingly, the system 400 may include a processor 402. Further, the processor 402 may be configured for obtaining one or more information associated with the creating of one or more tattoos on a portion of a skin of a body part of a client. Further, the processor 402 may be configured for analyzing the one or more information. Further, the processor 402 may be configured for determining one or more operations for the creating of the one or more tattoos based on the analyzing of the one or more information. Further, the processor 402 may be configured for generating one or more operation data associated with the one or more operations based on the determining of the one or more operations. Further, the system 400 may include a communication interface 404 communicatively coupled with the processor 402. Further, the communication interface 404 may be configured for transmitting the one or more operation data to one or more devices 406. Further, the one or more devices 406 may be configured for performing one or more device operations corresponding to the one or more operations for facilitating the creating of the one or more tattoos based on the one or more operation data.

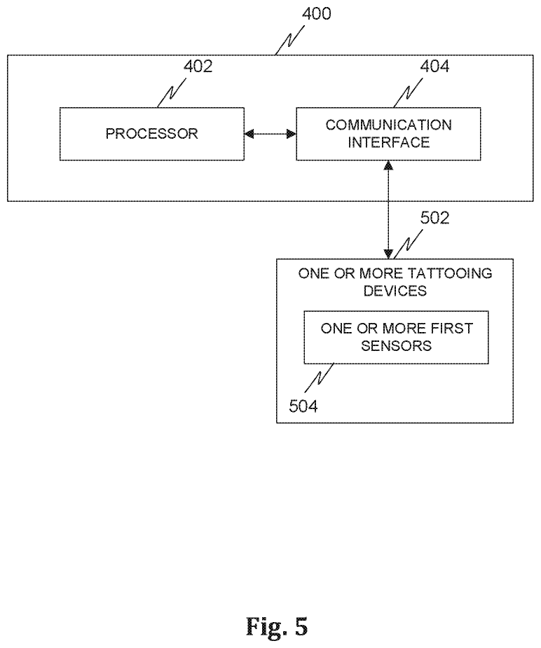

FIG. 5 illustrates a block diagram of the system 400 facilitating the management and creation of tattoos, in accordance with some embodiments. Further, in some embodiments, the one or more devices 406 include one or more tattooing devices 502. Further, the one or more device operations include one or more tattooing device operations associated with the one or more tattooing devices 502. Further, the one or more information includes one or more first sensor data representing one or more interaction conditions of the one or more tattooing devices. Further, the obtaining of the one or more information may include obtaining the one or more first sensor data from the one or more tattooing devices 502. Further, the one or more tattooing devices 502 include one or more first sensors 504 which may be configured for generating the one or more first sensor data. Further, the analyzing of the one or more information may include analyzing the one or more first sensor data. Further, the processor 402 may be further configured for identifying one or more skin interaction parameters based on the analyzing of the one or more first sensor data. Further, the processor 402 may be further configured for generating one or more control data for the one or more tattooing devices 502 based on the identifying of the one or more skin interaction parameters. Further, the one or more operation data includes one or more control data. Further, the performing of the one or more device operations includes performing the one or more tattooing device operations based on the one or more control data.

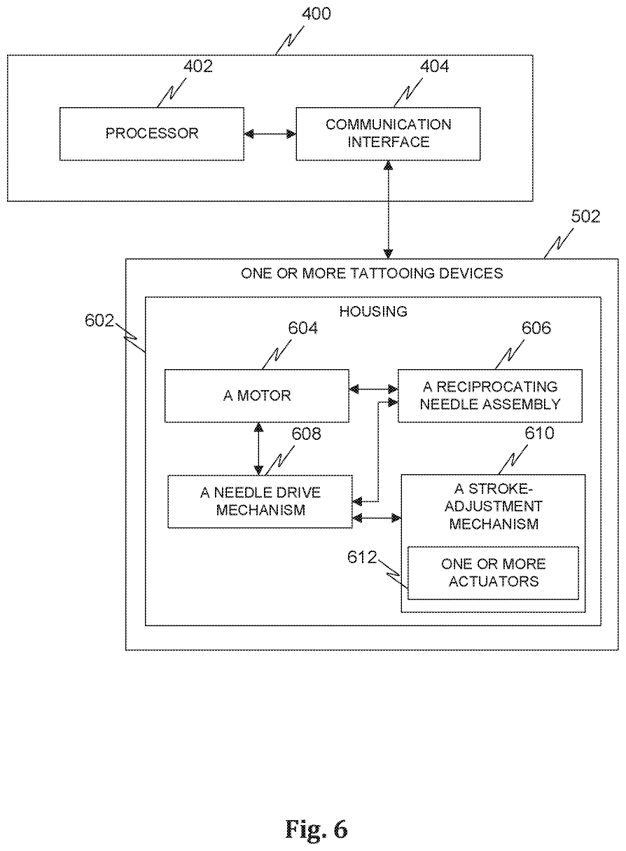

FIG. 6 illustrates a block diagram of the system 400 facilitating the management and creation of tattoos, in accordance with some embodiments. Further, in some embodiments, the one or more tattooing devices 502 include a housing 602. Further, the one or more tattooing devices 502 include a motor 604 disposed within the housing. Further, the one or more tattooing devices 502 include a reciprocating needle assembly 606 operably coupled with the motor 604. Further, the motor 604 may be configured for driving the reciprocating needle assembly 606. Further, the one or more tattooing devices 502 include a needle drive mechanism 608 mechanically coupled with the motor 604 and the reciprocating needle assembly 606. Further, the needle drive mechanism 608 may be configured for converting a motor motion of the motor 604 into a linear reciprocating motion of one or more tattooing needles of the reciprocating needle assembly 606. Further, the one or more tattooing devices 502 include a stroke-adjustment mechanism 610 mechanically coupled with the needle drive mechanism 608. Further, the one or more control data include one or more stroke control data. Further, the stroke-adjustment mechanism 610 includes one or more actuators 612 which may be configured for varying a stroke length of the linear reciprocating motion during the performing of the one or more tattooing device operations based on the one or more stroke control data.

Further, in some embodiments, the processor 402 may be further configured for determining one or more stroke length values for the reciprocating needle assembly based on the identifying of the one or more skin interaction parameters. Further, the processor 402 may be further configured for generating the one or more stroke control data based on the determining of the one or more stroke length values. Further, the generating of the one or more control data includes generating the one or more stroke control data.

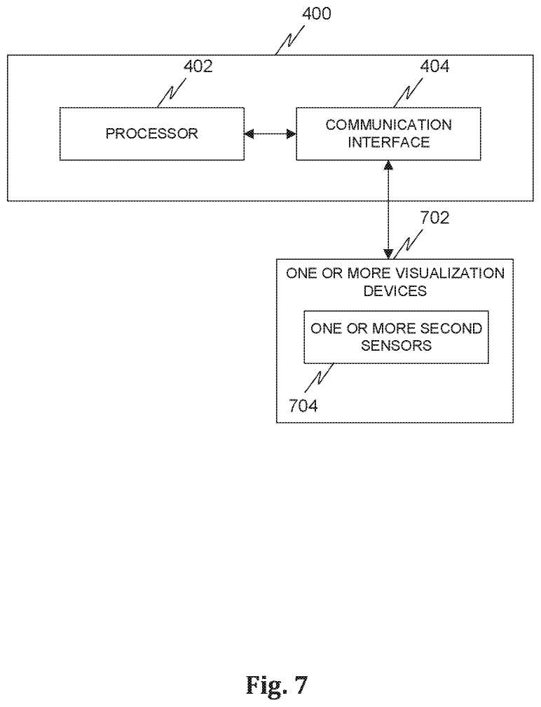

FIG. 7 illustrates a block diagram of the system 400 facilitating the management and creation of tattoos, in accordance with some embodiments. Further, in some embodiments, the one or more devices 406 include one or more visualization devices 702. Further, the one or more device operations include one or more visualization operations associated with the one or more visualization devices 702. Further, the one or more information includes one or more second sensor data representing one or more visual representations of the skin of the body part of the client. Further, the obtaining of the one or more information may include obtaining the one or more second sensor data from the one or more visualization devices 702. Further, the one or more visualization devices 702 include one or more second sensors 704 which may be configured for generating the one or more second sensor data. Further, the analyzing of the one or more information may include analyzing the one or more second sensor data. Further, the processor 402 may be further configured for determining one or more stencil data based on the analyzing of the one or more second sensor data. Further, the one or more stencil data represent one or more tattoo designs spatially aligned with the portion of the skin. Further, the processor 402 may be further configured for generating one or more display data for the one or more tattoo designs based on the determining of the one or more stencil data. Further, the one or more operation data include one or more display data. Further, the performing of the one or more device operations includes performing the one or more visualization operations based on the one or more display data.

Further, in some embodiments, the processor 402 may be further configured for generating one or more alignment data representing alignment of the one or more tattoo designs with the portion of the skin based on the one or more display data. Further, the processor 402 may be further configured for determining one or more anchoring data based on the one or more alignment data. Further, the processor 402 may be further configured for generating one or more updated display data based on the determining of the one or more anchoring data. Further, the one or more display data may include the one or more updated display data. Further, the performing of the one or more visualization operations includes maintaining the one or more tattoo designs in a positional alignment with the portion of the skin based on the one or more updated display data.

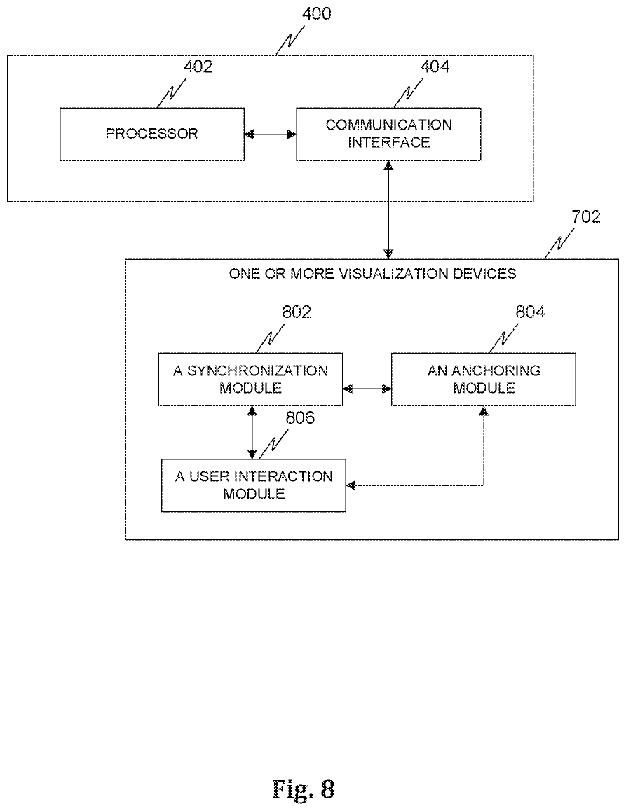

FIG. 8 illustrates a block diagram of the system 400 facilitating the management and creation of tattoos, in accordance with some embodiments. Further, in some embodiments, the each of the one or more visualization devices 702 may include a synchronization module 802 which may be configured for aligning the one or more tattoo designs with the portion of the skin based on the one or more alignment data. Further, the each of the one or more visualization devices 702 may include an anchoring module 804 which may be configured for maintaining the one or more tattoo designs in the positional alignment with the portion of the skin based on the one or more anchoring data.

Further, in some embodiments, the each of the one or more visualization devices 702 may include a user interaction module 806 which may be configured for generating one or more user interaction data based on one or more user interactions. Further, the communication interface 404 may be further configured for receiving the one or more user interaction data from the one or more visualization devices 702. Further, the processor 402 may be further configured for analyzing the one or more user interaction data. Further, the processor 402 may be further configured for determining one or more manipulation data based on the analyzing of the one or more user interaction data. Further, the processor 402 may be further configured for generating one or more updated stencil data based on the one or more stencil data and the one or more manipulation data. Further, the generating of the one or more updated display data may be further based on the one or more updated stencil data.

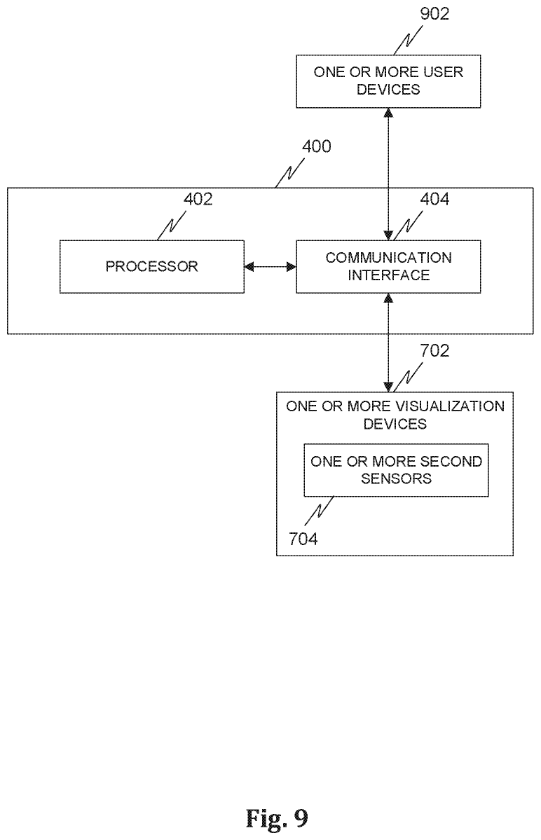

FIG. 9 illustrates a block diagram of the system 400 facilitating the management and creation of tattoos, in accordance with some embodiments. Further, in some embodiments, the communication interface 404 may be further configured for receiving one or more user input data from the one or more user devices 902. Further, the processor 402 may be further configured for analyzing the one or more user input data. Further, the generating of the one or more updated stencil data may be further based on the analyzing of the one or more user input data.

Further, in some embodiments, the obtaining of the one or more second sensor data may include obtaining the one or more second sensor data during a first time period. Further, the obtaining of the one or more information further may include obtaining one or more third sensor data. Further, the one or more second sensors 704 may be further configured for generating the one or more third sensor data in a second time period. Further, the second time period occurs later than the first time period. Further, the analyzing of the one or more information further may include analyzing the one or more third sensor data. Further, the processor 402 may be further configured for identifying one or more deformation data based on the analyzing of the one or more third sensor data. Further, the processor 402 may be further configured for determining one or more stencil compensation data based on the identifying of the one or more deformation data. Further, the generating of the one or more updated display data may be further based on the one or more stencil compensation data.

Further, in some embodiments, the one or more devices 406 further include the one or more tattooing devices 502. Further, the one or more device operations include one or more tattooing device operations associated with the one or more tattooing devices 502 relative to the one or more tattoo designs. Further, the performing of the one or more device operations may include performing the one or more tattooing device operations using the one or more tattooing devices 502. Further, the one or more display data include one or more execution path data representing one or more intended execution paths for the performing of the one or more tattooing device operations. Further, the one or more third sensor data include one or more actual execution path data representing one or more actual execution paths of the one or more tattooing devices 502 during the second time period. Further, the processor 402 may be further configured for analyzing the one or more actual execution path data and the one or more execution path data. Further, the processor 402 may be further configured for determining one or more path deviation data based on the analyzing of the one or more actual execution path data and the one or more execution path data. Further, the processor 402 may be further configured for generating one or more guidance data based on the determining of the one or more path deviation data. Further, the one or more updated display data may include the one or more guidance data.

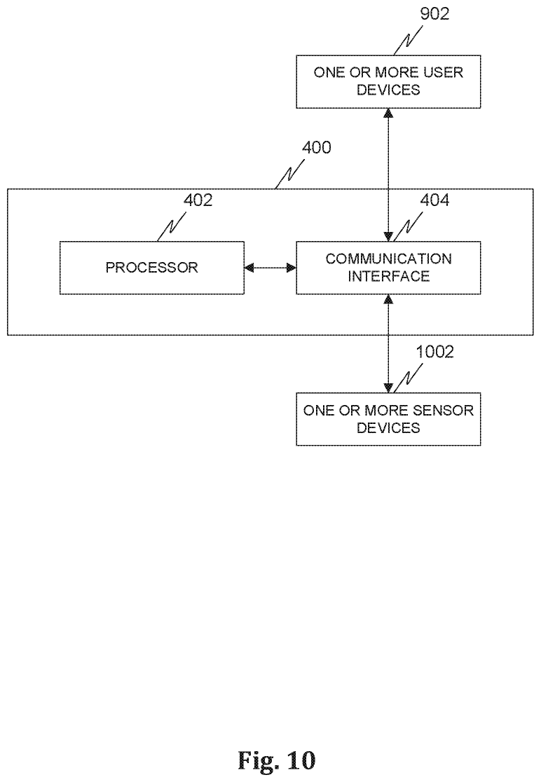

FIG. 10 illustrates a block diagram of the system 400 facilitating the management and creation of tattoos, in accordance with some embodiments. Further, in some embodiments, the one or more devices 406 include one or more sensor devices 1002. Further, the one or more device operations include one or more detection operations associated with the one or more sensor devices 1002. Further, the performing of the one or more device operations may include performing the one or more detection operations. Further, the one or more sensor devices 1002 may be configured for generating one or more tattooing condition data representing one or more tattooing conditions associated with the creating of the one or more tattoos based on the performing of the one or more detection operations. Further, the communication interface 404 may be further configured for receiving the one or more tattooing condition data from the one or more sensor devices 1002. Further, the communication interface 404 may be further configured for transmitting one or more alert data to the one or more user devices 902 associated with the one or more users. Further, the processor 402 may be further configured for analyzing the one or more tattooing condition data. Further, the processor 402 may be further configured for identifying one or more unsafe tattooing conditions based on the analyzing of the one or more tattooing condition data. Further, the processor 402 may be further configured for generating the one or more alert data based on the identifying of the one or more unsafe tattooing conditions.

In some embodiments, the analyzing of the one or more third sensor data includes analyzing the one or more third sensor data using one or more artificial intelligence (AI) models. Further, the identifying of the one or more deformation data includes identifying the one or more deformation data using the one or more AI models.

Further, in some embodiments, the one or more tattooing devices 502 may be associated with one or more operational parameters. Further, the performing of the one or more tattooing device operations may include performing the one or more tattooing device operations based on the one or more operational parameters during the first time period. Further, the one or more third sensor data include one or more ink saturation data representing one or more ink saturation levels relative to the portion of the skin during the second time period. Further, the analyzing of the one or more third sensor data may include analyzing the one or more ink saturation data. Further, the processor 402 may be further configured for determining one or more saturation abnormality data based on the analyzing of the one or more ink saturation data. Further, the processor 402 may be further configured for generating one or more updated operational parameters for the one or more tattooing devices 502 based on the determining of the one or more saturation abnormality data. Further, the performing of the one or more device operations includes performing the one or more tattooing device operations during the second time period based on the one or more updated operational parameters.

Further, in some embodiments, the one or more visualization operations include one or more training operations for training one or more users for the creating of the one or more tattoos. Further, the one or more display data include one or more guided tattoo execution overlay data representing one or more visual training instructions for the creating of the one or more tattoos. Further, the one or more information further includes one or more user action data representing one or more actions performed by the one or more users during the one or more training operations. Further, the processor 402 may be further configured for analyzing the one or more user action data. Further, the processor 402 may be further configured for determining one or more performance parameter data based on the analyzing of the one or more user action data. Further, the one or more performance parameter data may be associated with the one or more actions. Further, the processor 402 may be further configured for generating one or more performance score data based on the determining of the one or more performance parameter data. Further, the processor 402 may be further configured for generating one or more training feedback data based on the one or more performance score data. Further, the one or more display data include the one or more training feedback data.

Further, in some embodiments, the one or more information further includes one or more benchmark data representing one or more tattoo skill benchmarks. Further, the one or more user action data include one or more execution data associated with the one or more actions. Further, the processor 402 may be further configured for analyzing the one or more execution data. Further, the processor 402 may be further configured for comparing the one or more performance parameter data with the one or more benchmark data. Further, the processor 402 may be further configured for determining one or more skill level data based on the comparing of the one or more performance parameter data with the one or more benchmark data. Further, the processor 402 may be further configured for generating one or more credential data based on the determining of the one or more skill level data and the analyzing of the one or more execution data. Further, the one or more credential data indicate one or more skill levels of the one or more users.

Further, in some embodiments, the one or more second sensor data include one or more three-dimensional skin surface data representing one or more three-dimensional characteristics of the portion of the skin. Further, the analyzing of the one or more second sensor data may include analyzing the one or more three-dimensional skin surface data. Further, the processor 402 may be further configured for generating one or more skin model data based on the analyzing of the one or more three-dimensional skin surface data. Further, the processor 402 may be further configured for determining one or more skin property data based on the one or more skin model data. Further, the one or more skin property data represent one or more physical properties of the portion of the skin. Further, the processor 402 may be further configured for generating one or more simulated tattoo outcome data based on the one or more stencil data and the one or more skin property data. Further, the one or more simulated tattoo outcome data represent one or more simulated outcomes associated with the one or more tattoo designs. Further, the processor 402 may be further configured for generating one or more pre-execution visualization data based on the one or more simulated tattoo outcome data. Further, the one or more display data include the one or more pre-execution visualization data for visualizing the one or more tattoo designs relative to the portion of the skin.

Further, in some embodiments, the one or more tattooing condition data include one or more biological characteristic data representing one or more biological characteristics of the skin of the body part of the client. Further, the analyzing of the one or more tattooing condition data may include analyzing the one or more biological characteristic data. Further, the processor 402 may be further configured for determining one or more skin condition classification data based on the analyzing of the one or more biological characteristic data. Further, the one or more skin condition classification data represent one or more skin conditions of the portion of the skin. Further, the processor 402 may be further configured for determining one or more safe tattooing parameter data based on the determining of the one or more skin condition classification data. Further, the processor 402 may be further configured for generating one or more safety control data based on the one or more safe tattooing parameter data. Further, the one or more operation data include the one or more safety control data for facilitating the performing of the one or more device operations according to the one or more safe tattooing parameter data.

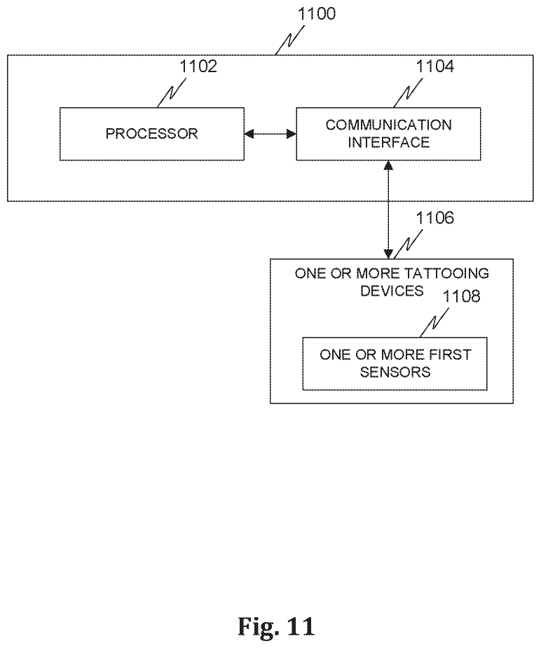

FIG. 11 illustrates a block diagram of a system 1100 facilitating the management and creation of tattoos, in accordance with some embodiments. Accordingly, the system 1100 may include a processor 1102. Further, the processor 1102 may be configured for obtaining one or more information associated with the creating of one or more tattoos on a portion of a skin of a body part of a client. Further, the one or more information includes one or more first sensor data representing one or more interaction conditions of one or more tattooing devices 1106. Further, the processor 1102 may be configured for analyzing the one or more information. Further, the analyzing of the one or more information includes analyzing the one or more first sensor data. Further, the processor 1102 may be configured for determining one or more operations for the creating of the one or more tattoos based on the analyzing of the one or more information. Further, the one or more operations include one or more tattooing device operations associated with the one or more tattooing devices 1106. Further, the processor 1102 may be configured for identifying one or more skin interaction parameters based on the analyzing of the one or more first sensor data. Further, the processor 1102 may be configured for generating one or more control data for the one or more tattooing devices 1106 based on the identifying of the one or more skin interaction parameters. Further, the processor 1102 may be configured for generating one or more operation data associated with the one or more operations based on the determining of the one or more operations. Further, the one or more operation data include the one or more control data. Further, the system 1100 may include a communication interface 1104 communicatively coupled with the processor 1102. Further, the communication interface 1104 may be configured for transmitting the one or more operation data to one or more devices. Further, the one or more devices include the one or more tattooing devices 1106. Further, the one or more tattooing devices 1106 include one or more first sensors 1108 which may be configured for generating the one or more first sensor data. Further, the one or more devices may be configured for performing one or more device operations corresponding to the one or more operations for facilitating the creating of the one or more tattoos based on the one or more operation data. Further, the one or more device operations include the one or more tattooing device operations. Further, the performing of the one or more device operations includes performing the one or more tattooing device operations based on the one or more control data.

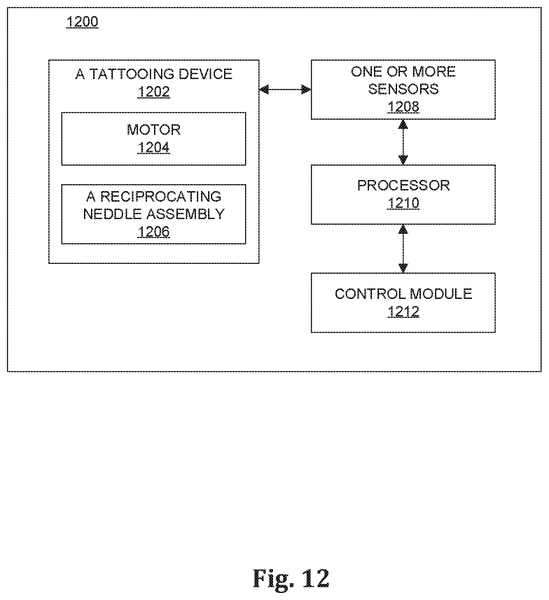

FIG. 12 illustrates block diagram of a tattooing system 1200, in accordance with some embodiments. Further, the tattooing system 1200 includes a tattooing device 1202 including a motor 1204 configured for driving a reciprocating needle assembly 1206. Further, the tattooing system 1200 includes one or more sensors 1208 configured for generating one or more sensor data representing one or more interaction conditions between the tattooing device 1202 and a skin surface of a body part of a client during performing one or more device operations. Further, the one or more interaction conditions include one or more of resistance, vibration, displacement, pressure, and motion. Further, the tattooing system 1200 includes a processor 1210 operatively coupled to the one or more sensors 1208. Further, the tattooing system 1200 includes a control module 1212 executed by the processor 1210. Further, the control module 1212 may be configured for receiving the one or more sensor data from the one or more sensors 1208 during the performing of the one or more device operations. Further, the control module 1212 may be configured for determining a change in the one or more interaction conditions based on the one or more sensor data. Further, the control module 1212 may be configured for automatically adjusting, in real time and without interrupting operation of the motor 1204, one or more operational parameters of the tattooing device 1202 in response to the change in the one or more interaction conditions. Further, the one or more operational parameters include one or more of stroke length, needle depth, motor speed, voltage, torque, and operating frequency.

In some embodiments, the present disclosure describes a system for facilitating the management and creation of tattoos. Accordingly, the system may include a processor. Further, the processor may be configured for obtaining one or more information associated with the creating of one or more tattoos on a portion of a skin of a body part of a client. Further, the one or more information includes one or more second sensor data representing one or more visual representations of the skin of the body part of the client. Further, the processor may be configured for analyzing the one or more information. Further, the analyzing of the one or more information includes analyzing the one or more second sensor data. Further, the processor may be configured for determining one or more operations for the creating of the one or more tattoos based on the analyzing of the one or more information. Further, the one or more operations include one or more visualization operations associated with one or more visualization devices. Further, the processor may be configured for determining one or more stencil data based on the analyzing of the one or more second sensor data. Further, the one or more stencil data represent one or more tattoo designs spatially aligned with the portion of the skin. Further, the processor may be configured for generating one or more display data for the one or more tattoo designs based on the determining of the one or more stencil data. Further, the processor may be configured for generating one or more operation data associated with the one or more operations based on the determining of the one or more operations. Further, the one or more operation data include the one or more display data. Further, the system may include a communication interface communicatively coupled with the processor. Further, the communication interface may be configured for transmitting the one or more operation data to one or more devices. Further, the one or more devices include the one or more visualization devices. Further, the one or more visualization devices include one or more second sensors which may be configured for generating the one or more second sensor data. Further, the one or more devices may be configured for performing one or more device operations corresponding to the one or more operations for facilitating the creating of the one or more tattoos based on the one or more operation data. Further, the one or more device operations include the one or more visualization operations. Further, the performing of the one or more device operations includes performing the one or more visualization operations based on the one or more display data.

In some embodiments, the one or more interaction conditions include one or more of position, orientation, displacement, vibration, pressure, resistance, torque, acceleration, and a skin-contact attribute associated with the one or more tattooing devices.

In some embodiments, the one or more first sensors 504 include one or more of a pressure sensor, a force sensor, a torque sensor, a vibration sensor, a displacement sensor, an accelerometer, a gyroscope, an inertial measurement unit, an optical sensor, and a skin-contact sensor which may be configured for generating the one or more first sensor data.

In some embodiments, the one or more skin interaction parameters include one or more of a skin resistance parameter, a skin contact parameter, a skin elasticity parameter, a needle penetration parameter, a vibration response parameter, a pressure response parameter, and a tissue interaction parameter associated with the portion of the skin.

In some embodiments, the one or more control data include one or more of a motor speed data, a voltage data, a torque data, a frequency data, a give data, a needle penetration depth data, a stroke length data, and an operating mode data for controlling the one or more tattooing devices.

In some embodiments, the motor motion includes a rotational motor motion generated by the motor. Further, the needle drive mechanism 608 includes one or more motion conversion structures which may be configured for converting the rotational motor motion into the linear reciprocating motion of the one or more tattooing needles.

In some embodiments, the stroke length includes a travel distance of the one or more tattooing needles along a needle travel axis during one cycle of the linear reciprocating motion. Further, the one or more actuators 612 may be configured for varying the travel distance based on the one or more stroke control data.

In some embodiments, the one or more visualization devices 702 include one or more of a wearable visualization device, a head-worn display device, an augmented reality display device, a mixed reality display device, a smart glasses device, and a display-enabled computing device which may be configured for performing the one or more visualization operations.

In some embodiments, the one or more visual representations include one or more of an image data, a video data, a depth data, a spatial mapping data, a surface topology data, an anatomical feature data, a curvature data, and landmark data associated with the skin of the body part of the client.

In some embodiments, the one or more second sensors 704 include one or more of an image sensor, a camera, a depth sensor, an infrared sensor, a structured-light sensor, a time-of-flight sensor, an optical tracking sensor, and an inertial sensor which may be configured for generating the one or more second sensor data.

In some embodiments, the one or more stencil data include one or more of a tattoo outline data, a tattoo design layer data, a tattoo scale data, a tattoo orientation data, a tattoo position data, a tattoo opacity data, a tattoo curvature data, and a tattoo registration data associated with the one or more tattoo designs.

In some embodiments, the one or more alignment data include one or more of a position alignment data, an orientation alignment data, a scale alignment data, a curvature alignment data, a landmark alignment data, and a surface mapping alignment data representing the alignment of the one or more tattoo designs with the portion of the skin.

In some embodiments, the one or more anchoring data include one or more of an anatomical landmark anchoring data, a skin feature anchoring data, a fiducial anchoring data, a contour anchoring data, and a mapped surface anchoring data for maintaining the one or more tattoo designs in the positional alignment with the portion of the skin.

In some embodiments, the synchronization module 802 may be configured for updating the alignment of the one or more tattoo designs based on changes in the one or more second sensor data. Further, the anchoring module 804 may be further configured for maintaining the positional alignment during movement of the skin of the body part of the client.

In some embodiments, the one or more user interactions include one or more of a gesture input, a voice input, a touch input, a button input, a remote input, a pointer input, and a gaze-based input associated with manipulation of the one or more tattoo designs.

In some embodiments, the one or more manipulation data include one or more of a translation data, a rotation data, a scaling data, a mirroring data, a warping data, an opacity adjustment data, a layering data, a segmentation data, and a curvature adaptation data associated with the one or more tattoo designs.

In some embodiments, the one or more third sensor data include a later-generated sensor data representing one or more of a movement data, a deformation data, a stretching data, a compression data, a breathing-induced movement data, a posture change data, and a localized skin displacement data associated with the portion of the skin during the second time period.

In some embodiments, the one or more deformation data include one or more of a skin stretch data, a skin compression data, a skin curvature change data, a skin displacement data, a surface topology change data, and an anatomical landmark movement data identified based on the analyzing of the one or more third sensor data.

In some embodiments, the one or more stencil compensation data include one or more of a stencil translation compensation data, a stencil rotation compensation data, a stencil scale compensation data, a stencil curvature compensation data, a stencil warping compensation data, and a stencil registration correction data for updating the one or more tattoo designs relative to the portion of the skin.

In some embodiments, the one or more execution path data include one or more of a line path data, a shading path data, a color packing path data, a stippling path data, a trajectory data, a sequence data, and a timing data representing the one or more intended execution paths.

In some embodiments, the one or more actual execution path data include one or more of an actual position data, an actual orientation data, an actual trajectory data, an actual speed data, an actual dwell time data, an actual directional change data, and an actual needle path data associated with movement of the one or more tattooing devices 502.

In some embodiments, the one or more path deviation data include one or more of a lateral deviation data, an angular deviation data, a depth deviation data, a timing deviation data, a trajectory deviation data, and a missed-area data representing a difference between the one or more actual execution paths and the one or more intended execution paths.

In some embodiments, the one or more guidance data include one or more of a visual cue data, a directional cue data, a depth warning data, a trajectory correction data, a missed-area indication data, an overwork warning data, and a haptic cue data associated with the one or more path deviation data.

In some embodiments, the one or more sensor devices 1002 include one or more of an optical sensor device, a spectral sensor device, a pressure sensor device, a temperature sensor device, a vibration sensor device, a skin-contact sensor device, and an environmental sensor device which may be configured for performing the one or more detection operations.

In some embodiments, the one or more tattooing condition data include one or more of a skin pressure data, a needle depth data, a vibration data, a resistance data, a temperature data, an ink saturation data, a skin inflammation data, a skin contact data, and an environmental condition data associated with the creating of the one or more tattoos.

In some embodiments, the one or more alert data include one or more of a visual alert data, an auditory alert data, a haptic alert data, an operational warning data, a safety threshold warning data, a skin trauma warning data, and a corrective instruction data associated with the one or more unsafe tattooing conditions.

In some embodiments, the one or more AI models include one or more of a computer vision model, a deformation detection model, a movement prediction model, a skin classification model, an ink saturation model, and a tattoo execution evaluation model which may be configured for analyzing the one or more third sensor data.

In some embodiments, the one or more operational parameters include one or more of stroke length, motor speed, voltage, torque, give, needle frequency, needle penetration depth, and ink delivery characteristic associated with operation of the one or more tattooing devices 502.

In some embodiments, the one or more updated operational parameters include one or more of an updated stroke length data, an updated motor speed data, an updated voltage data, an updated torque data, an updated give data, an updated needle frequency data, an updated penetration depth data, and an updated ink delivery data generated based on the one or more saturation abnormality data.

In some embodiments, the one or more user action data include one or more of a hand movement data, a tattooing device movement data, a timing data, a dwell time data, a stroke path data, a pressure data, a speed data, and a technique data associated with the one or more actions performed by the one or more users.

In some embodiments, the one or more performance parameter data include one or more of an accuracy data, a timing data, a technique data, a consistency data, a path-following data, a depth-control data, a motion-smoothness data, and a completion data associated with the one or more actions.

In some embodiments, the one or more benchmark data include one or more of a reference accuracy data, a reference timing data, a reference technique data, a reference path data, a reference pressure data, a reference speed data, and a reference completion data for evaluating the one or more actions performed by the one or more users.

In some embodiments, the one or more credential data include one or more of a certification data, a proficiency level data, a training completion data, a skill verification data, a score record data, and an authorization data indicating the one or more skill levels of the one or more users.

In some embodiments, the one or more three-dimensional characteristics include one or more of a surface curvature, a surface contour, a surface elevation, an anatomical curvature, a skin topography, a landmark position, and a surface deformation associated with the portion of the skin.

In some embodiments, the one or more skin property data include one or more of an elasticity data, a texture data, a hydration-related data, a pigmentation-related data, a curvature data, a thickness-related data, and a simulated tissue response data associated with the portion of the skin.

In some embodiments, the one or more simulated outcomes include one or more of an ink spread data, a color blending data, a saturation data, a fading data, a healing effect data, a scar risk data, and a long-term appearance data associated with the one or more tattoo designs.

In some embodiments, the one or more biological characteristic data include one or more of a skin thickness data, a hydration data, a vascularity data, a pigmentation data, a melanin density data, an inflammation data, an irritation data, and a skin sensitivity data associated with the skin of the body part of the client.

In some embodiments, the one or more safe tattooing parameter data include one or more of a safe needle depth data, a safe stroke length data, a safe motor speed data, a safe pressure data, a safe frequency data, a safe ink saturation data, and a safe operating threshold data associated with the one or more skin conditions.

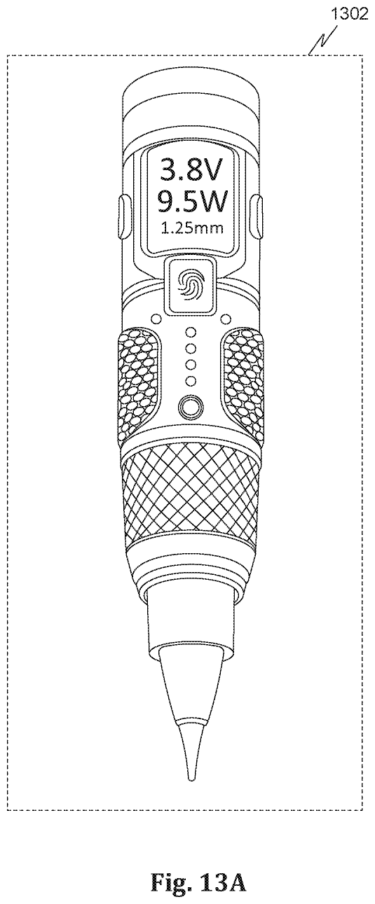

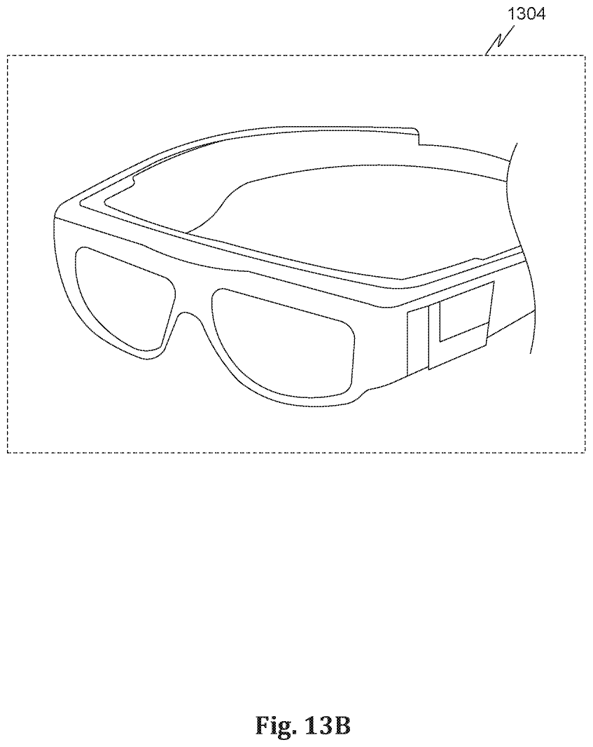

FIG. 13A and FIG. 13B illustrate one or more devices associated with the system 400, in accordance with some embodiments. Further, the one or more devices includes a tattooing device 1302 and a visualization device 1304.

In some embodiments, the one or more devices 406 may be and/or may include the one or more tattooing devices 502, the one or more visualization devices 702, the one or more sensor devices 1002, the one or more user devices 902, one or more computing devices, one or more remote computing systems, one or more cloud computing systems, one or more robotic systems, and/or any combination thereof. In some embodiments, the one or more devices may be configured to perform the one or more device operations based on the one or more operation data generated by the processor 402 and transmitted by the communication interface 404.

In some embodiments, the one or more tattooing devices 502 may be and/or may include the handheld tattoo device, the tattoo machine, the adaptive tattoo machine, the motor-assisted tattoo device, the robotic or semi-autonomous tattoo device, the tattooing instrument, and/or any compatible tattoo execution device. In some embodiments, the one or more tattooing devices 502 may include a housing, a motor, a reciprocating needle assembly, a needle drive mechanism, a stroke-adjustment mechanism, one or more actuators, one or more first sensors, and/or any combination thereof. In some embodiments, the one or more tattooing devices 502 may correspond to the handheld tattoo machine described as including the motor-driven reciprocating needle assembly, the linear or substantially linear needle drive mechanism, and the dynamically adjustable stroke-control mechanism.

In some embodiments, the motor may be configured to drive the reciprocating needle assembly. In some embodiments, the needle drive mechanism may be configured to convert the motor motion into the linear reciprocating motion and/or a substantially linear reciprocating motion of the one or more tattooing needles. In some embodiments, the stroke-adjustment mechanism may be and/or may include the dynamically adjustable stroke-control mechanism, the electronically controllable stroke-adjustment mechanism, the stroke adjustment actuator, the linear actuator, the solenoid, the piezoelectric device, the servo mechanism, the magnetic positioning assembly, the cam translation system, and/or any equivalent adjustment structure configured to vary the stroke length.

In some embodiments, the one or more stroke control data may be and/or may include the stroke adjustment data, the stroke length data, the stroke length adjustment data, the actuator control data, and/or the machine control data for causing the one or more actuators to vary the stroke length of the linear reciprocating motion. In some embodiments, the one or more stroke length values may define the travel distance of the one or more tattooing needles along the needle travel axis during one cycle of the linear reciprocating motion.

In some embodiments, the one or more first sensors 504 may be and/or may include the plurality of sensors, the one or more motion sensors, the one or more pressure sensors, the one or more force sensors, the one or more torque sensors, the one or more vibration sensors, the one or more displacement sensors, the one or more accelerometers, the one or more gyroscopes, the one or more inertial measurement units, the one or more optical sensors, the one or more skin-contact sensors, and/or any combination thereof. In some embodiments, the one or more first sensor data may be and/or may include sensor data, real-time sensor data, operational parameter data, motion data, displacement data, vibration data, torque data, resistance data, pressure data, acceleration data, orientation data, skin-contact data, needle excursion data, and/or any combination thereof.

In some embodiments, the one or more interaction conditions of the one or more tattooing devices 502 may be and/or may include the operational parameters, the skin-interaction parameters, the machine operating parameters, the position attribute, the orientation attribute, the displacement attribute, the vibration attribute, the pressure attribute, the resistance attribute, the torque attribute, the acceleration attribute, the skin-contact attribute, the needle penetration attribute, and/or any combination thereof.

In some embodiments, the one or more skin interaction parameters may be and/or may include the interaction conditions between the one or more tattooing devices 502 and the skin, the skin resistance parameter, the skin contact parameter, the skin elasticity parameter, the needle penetration parameter, the vibration response parameter, the pressure response parameter, the tissue interaction parameter, the ink deposition response parameter, and/or any combination thereof.

In some embodiments, the one or more control data may be and/or may include the machine control data, the tattooing device control data, the control instructions, the stroke control data, the motor speed data, the voltage data, the torque data, the frequency data, the give data, the needle penetration depth data, the operating mode data, the updated operational parameters, and/or any combination thereof. In some embodiments, the one or more operational parameters may be and/or may include stroke length, motor speed, voltage, torque, give, needle frequency, needle penetration depth, pressure, ink delivery characteristic, and/or any related tattooing parameter.

In some embodiments, the one or more visualization devices 702 may be and/or may include the wearable augmented reality glasses device, the wearable smart glasses, the augmented reality glasses, the wearable display, the head-mounted display, the head-worn display device, the augmented reality display device, the mixed reality display device, the smart glasses device, the display-enabled computing device, and/or any wearable visualization device configured to display one or more tattoo-related visual overlays. In some embodiments, the one or more visualization devices 702 may include one or more displays, one or more cameras, one or more depth sensors, one or more inertial sensors, one or more processors, one or more communication interfaces, and/or the one or more second sensors.

In some embodiments, the one or more visualization operations may be and/or may include displaying, rendering, projecting, overlaying, updating, manipulating, aligning, anchoring, maintaining, training, guiding, warning, and/or otherwise presenting the one or more tattoo designs, the one or more visual training instructions, the one or more guidance data, the one or more training feedback data, and/or the one or more pre-execution visualization data.

In some embodiments, the one or more second sensors 704 may be and/or may include the one or more cameras, the one or more imaging sensors, the one or more depth sensors, the one or more infrared sensors, the one or more structured-light sensors, the one or more time-of-flight sensors, the one or more optical tracking sensors, the one or more inertial sensors, and/or any sensor configured to generate the one or more second sensor data associated with the skin of the body part of the client.

In some embodiments, the one or more second sensor data may be and/or may include visual data, image data, video data, depth data, spatial mapping data, surface topology data, anatomical feature data, curvature data, landmark data, three-dimensional skin surface data, and/or any data representing the skin of the body part of the client. In some embodiments, the one or more visual representations may be and/or may include images, video frames, depth maps, surface maps, three-dimensional surface scans, skin topography maps, anatomical landmark representations, and/or curvature representations of the portion of the skin.

In some embodiments, the one or more stencil data may be and/or may include the digital stencil, the digital tattoo stencil, the projected digital stencil, the tattoo outline data, the tattoo design layer data, the tattoo scale data, the tattoo orientation data, the tattoo position data, the tattoo opacity data, the tattoo curvature data, the tattoo registration data, and/or any data representing the one or more tattoo designs. In some embodiments, the one or more tattoo designs may be spatially aligned with, visually aligned with, positionally aligned with, mapped onto, displayed relative to, and/or anchored relative to the portion of the skin.

In some embodiments, the one or more display data may be and/or may include the augmented reality display data, the stencil display data, the overlay data, the updated display data, the guided tattoo execution overlay data, the guidance data, the training feedback data, the pre-execution visualization data, the execution path data, and/or any data configured for causing the one or more visualization devices to display the one or more tattoo designs or related guidance relative to the portion of the skin.

In some embodiments, the one or more alignment data may be and/or may include the spatial registration data, the position alignment data, the orientation alignment data, the scale alignment data, the curvature alignment data, the landmark alignment data, and/or the surface mapping alignment data representing alignment of the one or more tattoo designs with the portion of the skin. In some embodiments, the synchronization module 802 may be configured to align, register, synchronize, recalibrate, and/or update the one or more tattoo designs relative to the portion of the skin based on the one or more alignment data.

In some embodiments, the one or more anchoring data may be and/or may include the anatomical landmark anchoring data, the skin feature anchoring data, the fiducial anchoring data, the contour anchoring data, the mapped surface anchoring data, and/or any anchoring data for maintaining the one or more tattoo designs in the positional alignment with the portion of the skin. In some embodiments, the anchoring module 804 may be configured to lock, maintain, preserve, update, and/or correct the positional alignment of the one or more tattoo designs during movement of the skin, breathing, posture change, deformation, stretching, compression, or localized displacement.

In some embodiments, the user interaction module 806 may be configured to generate the one or more user interaction data based on the one or more user interactions. In some embodiments, the one or more user interactions may be and/or may include gesture input, voice input, touch input, button input, remote input, pointer input, gaze-based input, physical control input, and/or any input associated with manipulating the one or more tattoo designs. In some embodiments, the one or more manipulation data may be and/or may include translation data, rotation data, scaling data, mirroring data, warping data, opacity adjustment data, layering data, segmentation data, curvature adaptation data, recall data, saving data, duplication data, and/or any manipulation-related data associated with the one or more tattoo designs.

In some embodiments, the one or more third sensor data may be and/or may include later-generated sensor data obtained after the one or more second sensor data. In some embodiments, the one or more third sensor data may represent movement data, deformation data, stretching data, compression data, breathing-induced movement data, posture change data, localized skin displacement data, actual execution path data, ink saturation data, and/or any sensor data generated during the second time period.

In some embodiments, the one or more deformation data may be and/or may include skin stretch data, skin compression data, skin curvature change data, skin displacement data, surface topology change data, anatomical landmark movement data, and/or any data identifying a change in the portion of the skin relative to a prior mapped condition. In some embodiments, the one or more stencil compensation data may be and/or may include stencil translation compensation data, stencil rotation compensation data, stencil scale compensation data, stencil curvature compensation data, stencil warping compensation data, stencil registration correction data, and/or any data for compensating the one or more tattoo designs based on the one or more deformation data.

In some embodiments, the one or more execution path data may be and/or may include intended execution path data, line path data, shading path data, color packing path data, stippling path data, trajectory data, sequence data, timing data, and/or any data representing intended movement of the one or more tattooing devices relative to the one or more tattoo designs. In some embodiments, the one or more actual execution path data may be and/or may include actual position data, actual orientation data, actual trajectory data, actual speed data, actual dwell time data, actual directional change data, actual needle path data, and/or any data associated with actual movement of the one or more tattooing devices.

In some embodiments, the one or more path deviation data may be and/or may include lateral deviation data, angular deviation data, depth deviation data, timing deviation data, trajectory deviation data, missed-area data, and/or any data representing a difference between the one or more actual execution paths and the one or more intended execution paths. In some embodiments, the one or more guidance data may be and/or may include visual cue data, directional cue data, depth warning data, trajectory correction data, missed-area indication data, overwork warning data, haptic cue data, audible cue data, and/or any corrective guidance data associated with the one or more path deviation data.

In some embodiments, the one or more sensor devices 1002 may be and/or may include the one or more optical sensor devices, the one or more spectral sensor devices, the one or more pressure sensor devices, the one or more temperature sensor devices, the one or more vibration sensor devices, the one or more skin-contact sensor devices, the one or more environmental sensor devices, and/or any sensor device configured to perform the one or more detection operations. In some embodiments, the one or more detection operations may include detecting, sensing, monitoring, measuring, classifying, validating, and/or generating the one or more tattooing condition data.

In some embodiments, the one or more tattooing condition data may be and/or may include skin pressure data, needle depth data, vibration data, resistance data, temperature data, ink saturation data, skin inflammation data, skin contact data, environmental condition data, biological characteristic data, and/or any data representing a condition associated with the creating of the one or more tattoos. In some embodiments, the one or more unsafe tattooing conditions may include excessive pressure, excessive needle depth, excessive vibration, excessive resistance, excessive temperature, over-saturation, under-saturation, inflammation, irritation, loss of skin contact, excessive skin trauma risk, and/or any condition exceeding one or more safe tattooing thresholds.

In some embodiments, the one or more alert data may be and/or may include visual alert data, auditory alert data, haptic alert data, operational warning data, safety threshold warning data, skin trauma warning data, corrective instruction data, and/or any alert data associated with the one or more unsafe tattooing conditions.

In some embodiments, the one or more artificial intelligence models may be and/or may include the machine learning models, the computer vision models, the deformation detection models, the movement prediction models, the skin classification models, the ink saturation models, the tattoo execution evaluation models, the tattooing technique classification models, the predictive analytics models, and/or any artificial intelligence processing model configured to analyze the one or more information.

In some embodiments, the one or more ink saturation data may represent one or more ink saturation levels, ink deposition levels, pigment density levels, under-saturation conditions, over-saturation conditions, ink absorption characteristics, ink dispersion characteristics, and/or pigmentation response associated with the portion of the skin. In some embodiments, the one or more saturation abnormality data may represent over-saturation, under-saturation, non-uniform saturation, insufficient pigment density, excessive pigment density, and/or any ink deposition abnormality. In some embodiments, the one or more updated operational parameters may be generated to adjust stroke length, motor speed, voltage, torque, give, needle frequency, needle penetration depth, pressure, and/or ink delivery characteristic.

In some embodiments, the one or more training operations may be and/or may include augmented reality training operations, guided tattoo execution operations, simulated tattooing operations, performance evaluation operations, certification operations, and/or any operation for training the one or more users for the creating of the one or more tattoos. In some embodiments, the one or more guided tattoo execution overlay data may be and/or may include visual training instruction data, stroke path overlay data, depth instruction data, timing instruction data, technique instruction data, correction overlay data, and/or any data displayed for training the one or more users.

In some embodiments, the one or more user action data may be and/or may include hand movement data, tattooing device movement data, timing data, dwell time data, stroke path data, pressure data, speed data, technique data, and/or any data associated with actions performed by the one or more users. In some embodiments, the one or more performance parameter data may be and/or may include accuracy data, timing data, technique data, consistency data, path-following data, depth-control data, motion-smoothness data, completion data, and/or any objective performance metric associated with the one or more actions.

In some embodiments, the one or more benchmark data may be and/or may include reference accuracy data, reference timing data, reference technique data, reference path data, reference pressure data, reference speed data, reference completion data, tattoo skill benchmark data, and/or benchmark standard data for evaluating the one or more actions. In some embodiments, the one or more credential data may be and/or may include certification data, proficiency level data, training completion data, skill verification data, score record data, authorization data, and/or any data indicating the one or more skill levels of the one or more users.

In some embodiments, the one or more three-dimensional skin surface data may be generated from the one or more second sensors and may represent surface curvature, surface contour, surface elevation, anatomical curvature, skin topography, landmark position, surface deformation, and/or three-dimensional characteristics of the portion of the skin. In some embodiments, the one or more skin model data may be and/or may include the digital model, the three-dimensional model, the digital twin, the skin digital twin, the simulated skin surface model, and/or any data model representing the portion of the skin.

In some embodiments, the one or more skin property data may be and/or may include physical property data, biological property data, elasticity data, texture data, hydration-related data, pigmentation-related data, curvature data, thickness-related data, simulated tissue response data, and/or any data representing properties of the portion of the skin. In some embodiments, the one or more simulated tattoo outcome data may be and/or may include ink spread data, color blending data, saturation data, fading data, healing effect data, scar risk data, long-term appearance data, and/or any simulated outcome associated with the one or more tattoo designs.

In some embodiments, the one or more pre-execution visualization data may be and/or may include data for displaying, before performing the one or more tattooing device operations, one or more predicted tattoo outcomes, one or more simulated healed outcomes, one or more simulated color blending outcomes, one or more simulated ink spread outcomes, one or more simulated fading outcomes, and/or one or more optimized tattoo placement outcomes relative to the portion of the skin.

In some embodiments, the one or more biological characteristic data may be and/or may include skin thickness data, hydration data, vascularity data, pigmentation data, melanin density data, inflammation data, irritation data, skin sensitivity data, and/or any biological characteristic of the skin of the body part of the client. In some embodiments, the one or more skin condition classification data may be and/or may include skin type data, hydration classification data, elasticity classification data, vascularity classification data, pigmentation classification data, inflammation classification data, irritation classification data, and/or any classification of the one or more skin conditions.

In some embodiments, the one or more safe tattooing parameter data may be and/or may include safe needle depth data, safe stroke length data, safe motor speed data, safe pressure data, safe frequency data, safe ink saturation data, safe operating threshold data, and/or any parameter data for reducing risk of damage to the skin. In some embodiments, the one or more safety control data may be and/or may include the one or more control data, the one or more updated operational parameters, one or more alert-triggering data, one or more halt-operation data, one or more parameter-limiting data, and/or one or more safety-based operation data configured to cause the one or more devices to perform the one or more device operations according to the one or more safe tattooing parameter data.

In some embodiments, the one or more user devices 902 may be and/or may include one or more mobile devices, one or more tablet devices, one or more desktop computing devices, one or more wearable computing devices, one or more remote computing devices, one or more cloud-connected devices, and/or one or more input devices associated with the one or more users. In some embodiments, the one or more users may include an artist, an operator, a trainee, an instructor, a remote expert, a supervisor, and/or any person interacting with the system.

In some embodiments, the one or more operations may be and/or may include tattooing device operations, visualization operations, detection operations, training operations, safety operations, alignment operations, anchoring operations, manipulation operations, compensation operations, guidance operations, simulation operations, certification operations, and/or any operation associated with facilitating the creating of the one or more tattoos. In some embodiments, the one or more operation data may be and/or may include the one or more control data, the one or more display data, the one or more updated display data, the one or more guidance data, the one or more alert data, the one or more safety control data, the one or more training feedback data, the one or more credential data, the one or more pre-execution visualization data, and/or any data for causing the one or more devices to perform the one or more device operations.

In some embodiments, the communication interface 404 may be and/or may include one or more wired communication interfaces, one or more wireless communication interfaces, one or more network interfaces, one or more short-range communication interfaces, one or more encrypted communication interfaces, one or more cloud communication interfaces, and/or one or more device-to-device communication interfaces. In some embodiments, communication between the processor 402, the one or more tattooing devices 502, the one or more visualization devices 702, the one or more sensor devices 1002, and/or the one or more user devices 406 may be encrypted.

In some embodiments, the system 400 may operate in a closed-loop configuration in which the processor 402 obtains the one or more information from the one or more devices 406, analyzes the one or more information, generates the one or more operation data, and transmits the one or more operation data to the one or more devices 406 for causing adaptive control, visualization, safety monitoring, guidance, training, simulation, and/or tattoo execution. In some embodiments, the closed-loop configuration may integrate tattooing device control with augmented reality guidance and sensor-based feedback.

Although the invention has been explained in relation to its preferred embodiment, it is to be understood that many other possible modifications and variations can be made without departing from the spirit and scope of the invention as hereinafter claimed.

What is claimed is:

1. A system for facilitating managing creating tattoos, the system comprising:
a processor configured for:
obtaining one or more information associated with the creating of one or more tattoos on a portion of a skin of a body part of a client;
analyzing the one or more information;
determining one or more operations for the creating of the one or more tattoos based on the analyzing of the one or more information; and
generating one or more operation data associated with the one or more operations based on the determining of the one or more operations; and
a communication interface communicatively coupled with the processor, wherein the communication interface is configured for transmitting the one or more operation data to one or more devices, wherein the one or more devices are configured for performing one or more device operations corresponding to the one or more operations for facilitating the creating of the one or more tattoos based on the one or more operation data.

2. The system of claim 1, wherein the one or more devices comprise one or more tattooing devices, wherein the one or more device operations comprise one or more tattooing device operations associated with the one or more tattooing devices, wherein the one or more information comprise one or more first sensor data representing one or more interaction conditions of the one or more tattooing devices, wherein the obtaining of the one or more information comprises obtaining the one or more first sensor data from the one or more tattooing devices, wherein the one or more tattooing devices comprise one or more first sensors configured for generating the one or more first sensor data, wherein the analyzing of the one or more information comprises analyzing the one or more first sensor data, wherein the processor is further configured for:
identifying one or more skin interaction parameters based on the analyzing of the one or more first sensor data; and
generating one or more control data for the one or more tattooing devices based on the identifying of the one or more skin interaction parameters, wherein the one or more operation data comprises one or more control data, wherein the performing of the one or more device operations comprises performing the one or more tattooing device operations based on the one or more control data.

3. The system of claim 2, wherein the one or more tattooing devices comprise:
a housing;
a motor disposed within the housing;
a reciprocating needle assembly operably coupled with the motor, wherein the motor is configured for driving the reciprocating needle assembly;
a needle drive mechanism mechanically coupled with the motor and the reciprocating needle assembly, wherein the needle drive mechanism is configured for converting a motor motion of the motor into a linear reciprocating motion of one or more tattooing needles of the reciprocating needle assembly; and
a stroke-adjustment mechanism mechanically coupled with the needle drive mechanism, wherein the one or more control data comprise one or more stroke control data, wherein the stroke-adjustment mechanism comprises one or more actuators configured for varying a stroke length of the linear reciprocating motion during the performing of the one or more tattooing device operations based on the one or more stroke control data.

4. The system of claim 3, wherein the processor is further configured for:
determining one or more stroke length values for the reciprocating needle assembly based on the identifying of the one or more skin interaction parameters; and
generating the one or more stroke control data based on the determining of the one or more stroke length values, wherein the generating of the one or more control data comprises generating the one or more stroke control data.

5. The system of claim 1, wherein the one or more devices comprise one or more visualization devices, wherein the one or more device operations comprise one or more visualization operations associated with the one or more visualization devices, wherein the one or more information comprise one or more second sensor data representing one or more visual representations of the skin of the body part of the client, wherein the obtaining of the one or more information comprises obtaining the one or more second sensor data from the one or more visualization devices, wherein the one or more visualization devices comprise one or more second sensors configured for generating the one or more second sensor data, wherein the analyzing of the one or more information comprises analyzing the one or more second sensor data, wherein the processor is further configured for:
determining one or more stencil data based on the analyzing of the one or more second sensor data, wherein the one or more stencil data represent one or more tattoo designs spatially aligned with the portion of the skin; and
generating one or more display data for the one or more tattoo designs based on the determining of the one or more stencil data, wherein the one or more operation data comprise one or more display data, wherein the performing of the one or more device operations comprises performing the one or more visualization operations based on the one or more display data.

6. The system of claim 5, wherein the processor is further configured for:
generating one or more alignment data representing alignment of the one or more tattoo designs with the portion of the skin based on the one or more display data;
determining one or more anchoring data based on the one or more alignment data; and
generating one or more updated display data based on the determining of the one or more anchoring data, wherein the one or more display data comprise the one or more updated display data, wherein the performing of the one or more visualization operations comprises maintaining the one or more tattoo designs in a positional alignment with the portion of the skin based on the one or more updated display data.

7. The system of claim 6, wherein each of the one or more visualization devices comprises:
a synchronization module configured for aligning the one or more tattoo designs with the portion of the skin based on the one or more alignment data; and
an anchoring module configured for maintaining the one or more tattoo designs in the positional alignment with the portion of the skin based on the one or more anchoring data.

8. The system of claim 6, wherein each of the one or more visualization devices comprises a user interaction module configured for generating one or more user interaction data based on one or more user interactions, wherein the communication interface is further configured for receiving the one or more user interaction data from the one or more visualization devices, wherein the processor is further configured for:
analyzing the one or more user interaction data;
determining one or more manipulation data based on the analyzing of the one or more user interaction data; and
generating one or more updated stencil data based on the one or more stencil data and the one or more manipulation data, wherein the generating of the one or more updated display data is further based on the one or more updated stencil data.

9. The system of claim 8, wherein the communication interface is further configured for receiving one or more user input data from one or more user devices associated with one or more users, wherein the processor is further configured for analyzing the one or more user input data, wherein the generating of the one or more updated stencil data is further based on the analyzing of the one or more user input data.

10. The system of claim 6, wherein the obtaining of the one or more second sensor data comprises obtaining the one or more second sensor data during a first time period, wherein the obtaining of the one or more information further comprises obtaining one or more third sensor data, wherein the one or more second sensors are further configured for generating the one or more third sensor data in a second time period, wherein the second time period occurs later than the first time period, wherein the analyzing of the one or more information further comprises analyzing the one or more third sensor data, wherein the processor is further configured for:
identifying one or more deformation data based on the analyzing of the one or more third sensor data; and
determining one or more stencil compensation data based on the identifying of the one or more deformation data, wherein the generating of the one or more updated display data is further based on the one or more stencil compensation data.

11. The system of claim 10, wherein the one or more devices further comprise one or more tattooing devices, wherein the one or more device operations comprise one or more tattooing device operations associated with the one or more tattooing devices relative to the one or more tattoo designs, wherein the performing of the one or more device operations comprises performing the one or more tattooing device operations using the one or more tattooing devices, wherein the one or more display data comprise one or more execution path data representing one or more intended execution paths for the performing of the one or more tattooing device operations, wherein the one or more third sensor data comprise one or more actual execution path data representing one or more actual execution paths of the one or more tattooing devices during the second time period, wherein the processor is further configured for:
analyzing the one or more actual execution path data and the one or more execution path data;
determining one or more path deviation data based on the analyzing of the one or more actual execution path data and the one or more execution path data; and
generating one or more guidance data based on the determining of the one or more path deviation data, wherein the one or more updated display data comprise the one or more guidance data.

12. The system of claim 1, wherein the one or more devices comprise one or more sensor devices, wherein the one or more device operations comprise one or more detection operations associated with the one or more sensor devices, wherein the performing of the one or more device operations comprises performing the one or more detection operations, wherein the one or more sensor devices are configured for generating one or more tattooing condition data representing one or more tattooing conditions associated with the creating of the one or more tattoos based on the performing of the one or more detection operations, wherein the communication interface is further configured for:
receiving the one or more tattooing condition data from the one or more sensor devices; and
transmitting one or more alert data to one or more user devices associated with one or more users, wherein the processor is further configured for:
analyzing the one or more tattooing condition data;
identifying one or more unsafe tattooing conditions based on the analyzing of the one or more tattooing condition data; and
generating the one or more alert data based on the identifying of the one or more unsafe tattooing conditions.

13. The system of claim 10, wherein the analyzing of the one or more third sensor data comprises analyzing the one or more third sensor data using one or more artificial intelligence (AI) models, wherein the identifying of the one or more deformation data comprises identifying the one or more deformation data using the one or more AI models.

14. The system of claim 11, wherein the one or more tattooing devices are associated with one or more operational parameters, wherein the performing of the one or more tattooing device operations comprises performing the one or more tattooing device operations based on the one or more operational parameters during the first time period, wherein the one or more third sensor data comprise one or more ink saturation data representing one or more ink saturation levels relative to the portion of the skin during the second time period, wherein the analyzing of the one or more third sensor data comprises analyzing the one or more ink saturation data, wherein the processor is further configured for:
determining one or more saturation abnormality data based on the analyzing of the one or more ink saturation data; and
generating one or more updated operational parameters for the one or more tattooing devices based on the determining of the one or more saturation abnormality data, wherein the performing of the one or more device operations comprises performing the one or more tattooing device operations during the second time period based on the one or more updated operational parameters.

15. The system of claim 11, wherein the one or more visualization operations comprise one or more training operations for training one or more users for the creating of the one or more tattoos, wherein the one or more display data comprise one or more guided tattoo execution overlay data representing one or more visual training instructions for the creating of the one or more tattoos, wherein the one or more information further comprise one or more user action data representing one or more actions performed by the one or more users during the one or more training operations, wherein the processor is further configured for:
analyzing the one or more user action data;
determining one or more performance parameter data based on the analyzing of the one or more user action data, wherein the one or more performance parameter data are associated with the one or more actions;
generating one or more performance score data based on the determining of the one or more performance parameter data; and
generating one or more training feedback data based on the one or more performance score data, wherein the one or more display data comprise the one or more training feedback data.

16. The system of claim 15, wherein the one or more information further comprise one or more benchmark data representing one or more tattoo skill benchmarks, wherein the one or more user action data comprise one or more execution data associated with the one or more actions, wherein the processor is further configured for:
analyzing the one or more execution data;
comparing the one or more performance parameter data with the one or more benchmark data;
determining one or more skill level data based on the comparing of the one or more performance parameter data with the one or more benchmark data; and
generating one or more credential data based on the determining of the one or more skill level data and the analyzing of the one or more execution data, wherein the one or more credential data indicate one or more skill levels of the one or more users.

17. The system of claim 5, wherein the one or more second sensor data comprise one or more three-dimensional skin surface data representing one or more three-dimensional characteristics of the portion of the skin, wherein the analyzing of the one or more second sensor data comprises analyzing the one or more three-dimensional skin surface data, wherein the processor is further configured for:
generating one or more skin model data based on the analyzing of the one or more three-dimensional skin surface data, wherein the one or more skin model data represent one or more three-dimensional models of the portion of the skin;
determining one or more skin property data based on the one or more skin model data, wherein the one or more skin property data represent one or more physical properties of the portion of the skin;
generating one or more simulated tattoo outcome data based on the one or more stencil data and the one or more skin property data, wherein the one or more simulated tattoo outcome data represent one or more simulated outcomes associated with the one or more tattoo designs; and
generating one or more pre-execution visualization data based on the one or more simulated tattoo outcome data, wherein the one or more display data comprise the one or more pre-execution visualization data for visualizing the one or more tattoo designs relative to the portion of the skin.

18. The system of claim 12, wherein the one or more tattooing condition data comprise one or more biological characteristic data representing one or more biological characteristics of the skin of the body part of the client, wherein the analyzing of the one or more tattooing condition data comprises analyzing the one or more biological characteristic data, wherein the processor is further configured for:
determining one or more skin condition classification data based on the analyzing of the one or more biological characteristic data, wherein the one or more skin condition classification data represent one or more skin conditions of the portion of the skin;
determining one or more safe tattooing parameter data based on the determining of the one or more skin condition classification data; and
generating one or more safety control data based on the one or more safe tattooing parameter data, wherein the one or more operation data comprise the one or more safety control data for facilitating the performing of the one or more device operations according to the one or more safe tattooing parameter data.

19. A system for facilitating managing creating tattoos, the system comprising:
a processor configured for:
obtaining one or more information associated with the creating of one or more tattoos on a portion of a skin of a body part of a client, wherein the one or more information comprises one or more first sensor data representing one or more interaction conditions of one or more tattooing devices;
analyzing the one or more information, wherein the analyzing of the one or more information comprises analyzing the one or more first sensor data;
determining one or more operations for the creating of the one or more tattoos based on the analyzing of the one or more information, wherein the one or more operations comprise one or more tattooing device operations associated with the one or more tattooing devices;
identifying one or more skin interaction parameters based on the analyzing of the one or more first sensor data;
generating one or more control data for the one or more tattooing devices based on the identifying of the one or more skin interaction parameters; and
generating one or more operation data associated with the one or more operations based on the determining of the one or more operations, wherein the one or more operation data comprise the one or more control data; and
a communication interface communicatively coupled with the processor, wherein the communication interface is configured for transmitting the one or more operation data to one or more devices, wherein the one or more devices comprise the one or more tattooing devices, wherein the one or more tattooing devices comprise one or more first sensors configured for generating the one or more first sensor data, wherein the one or more devices are configured for performing one or more device operations corresponding to the one or more operations for facilitating the creating of the one or more tattoos based on the one or more operation data, wherein the one or more device operations comprise the one or more tattooing device operations, wherein the performing of the one or more device operations comprises performing the one or more tattooing device operations based on the one or more control data.

20. A tattooing system comprising:
a tattooing device including a motor configured for driving a reciprocating needle assembly;
one or more sensors configured for generating one or more sensor data representing one or more interaction conditions between the tattooing device and a skin surface of a body part of a client during performing one or more device operations, wherein the one or more interaction conditions comprise one or more of resistance, vibration, displacement, pressure, and motion;
a processor operatively coupled to the one or more sensors; and
a control module executed by the processor, wherein the control module is configured for:
receiving the one or more sensor data from the one or more sensors during the performing of the one or more device operations;
determining a change in the one or more interaction conditions based on the sensor data; and
automatically adjusting, in real time and without interrupting operation of the motor, one or more operational parameters of the tattooing device in response to the change in the one or more interaction conditions, wherein the one or more operational parameters comprise one or more of stroke length, needle depth, motor speed, voltage, torque, and operating frequency.
